(19) United States
(12) Patent Application Publication
(10) Pub. No.: US 2026/0069760 A1
(43) Pub. Date: Mar. 12, 2026

(54) VACUUM BASED SYSTEM AND METHOD FOR TREATING A GASTROINTESTINAL TRACT OF A SUBJECT

(71) Applicant: THERADRAIN Ltd., Ra'anana (IL)

(72) Inventors: Michael Bortz, Kfar Saba (IL); Adam Sagiv, Bnai Atarot (IL); Oded Meiri, Ram On (IL); Petros Benias, Englewood Cliffs, NJ (US); Mitchell Roslin, Armonk, NY (US)

(21) Appl. No.: 19/395,704

(22) Filed: Nov. 20, 2025

Related U.S. Application Data
(63) Continuation of application No. PCT/IB2024/057179, filed on Jul. 24, 2024.
(60) Provisional application No. 63/635,451, filed on Apr. 17, 2024.

(51) Int. Cl. A61M 1/00; A61M 25/00

(52) U.S. Cl. CPC A61M 1/87; A61M 25/007; A61M 25/0074; A61M 2210/105

(57) ABSTRACT

A medical system for applying negative pressure within a gastrointestinal tract of a subject, including a linearizing element, an elongate tube, and a fluid-tight lumen. The elongate tube includes a channel along a longitudinal portion, at least one portal in fluid communication with the channel, and a shape-forming wire extending along a longitudinal length of the elongate tube. The elongate tube has a delivery state when associated with the linearizing element and a first operative state when dissociated from the linearizing element, in which the elongate tube forms a coil. A rotational orientation of the shape-forming wire relative to the elongate tube is fixed throughout an entire length of the shape-forming wire. The fluid-tight lumen is in fluid communication with the elongate tube, and is adapted to couple to a source of negative pressure and to deliver negative pressure to the elongate tube.

### CROSS-REFERENCE TO RELATED APPLICATIONS

The present application is a continuation of PCT/IB2024/057179 filed on Jul. 24, 2024, which is incorporated by referenced as if fully set forth herein. PCT/IB2024/057179 gains priority from U.S. Provisional Patent Application No. 63/635,451, filed on Apr. 17, 2024, which is incorporated by referenced as if fully set forth herein.

### BACKGROUND

Wounds in the gastrointestinal tract such as perforations and post-surgical leaks, and particularly in the esophagus, are common in endoscopic and open surgical procedures. The endoluminal location of these wounds and natural wet environment surrounding the wounds make these wounds particularly difficult to treat. Limited treatment options exist for these wounds which have significant morbidity and mortality rates while also involving extensive hospital stay.

Vacuum assisted closure (VAC) therapy can increase the rate of wound closure. Negative pressure wound therapy (NPWT) or VAC therapy is the application of sub-atmospheric pressure to acute or chronic wounds to promote the healing of a wound. In theory, creating a negative-pressure in the local wound environment, draws away bacteria, exudate, fluid, and debris tissue from the wound site, increases the rate of healing by promoting blood flow and facilitates localized cell migration and proliferation.

There is a need for improved techniques and devices for assisting in healing of wounds in the GI tract, and particularly in the esophagus, by applying negative pressure to the vicinity of the wounds.

### SUMMARY

Various applications herein relate to medical systems and methods for removal of liquid from a target area in the GI tract, for example to assist in healing of an extraluminal or endoluminal wounds.

In accordance with an embodiment of the disclosed technology, there is provided a medical system for applying negative pressure within a gastrointestinal tract of a subject. The medical system includes linearizing element, an elongate tube, and a fluid-tight lumen.

The elongate tube includes at least one channel along at least a longitudinal portion of the elongate tube, and at least one portal in fluid communication with the at least one channel. The elongate tube further includes a shape-forming wire fixed to or embedded within the elongate tube and extending along a longitudinal length thereof.

The elongate tube has a delivery state when associated with the linearizing element and a first operative state when dissociated from the linearizing element. In the first operative state, the elongate tube forms a coil including a plurality of loops, the coil having an axial length (L) of at least 15 mm and the plurality of loops including at least four loops. The elongate tube has a first flexure modulus in a coil-radial direction of the elongate tube and a second flexure modulus in a coil-axial direction of the elongate tube, the second flexure modulus being greater than the first flexure modulus.

The fluid-tight lumen is in fluid communication with a first end of the elongate tube, and is adapted to couple to a source of negative pressure and to deliver negative pressure to the elongate tube via the first end of the elongate tube.

In accordance with another embodiment of the disclosed technology, there is provided a medical system for applying negative pressure within a gastrointestinal tract of a subject. The medical system includes linearizing element, an elongate tube, and a fluid-tight lumen.

The elongate tube includes at least one channel along at least a longitudinal portion of the elongate tube, and at least one portal in fluid communication with the at least one channel. The elongate tube further includes a shape-forming wire fixed to or embedded within the elongate tube and extending along a longitudinal length thereof.

The elongate tube has a delivery state when associated with the linearizing element and a first operative state when dissociated from the linearizing element. In the first operative state, the elongate tube forms a coil including a plurality of loops, the coil having an axial length (L) of at least 15 mm and the plurality of loops including at least four loops. A position of the shape-forming wire within the elongate tube and the cross-sectional shape of the shape-forming wire are such that the elongate tube has a first flexure modulus in a coil-radial direction of the elongate tube and a second flexure modulus in a coil-axial direction of the elongate tube, the second flexure modulus being greater than the first flexure modulus.

The fluid-tight lumen is in fluid communication with a first end of the elongate tube, and is adapted to couple to a source of negative pressure and to deliver negative pressure to the elongate tube via the end of the elongate tube.

The fluid-tight lumen includes a tube formed of a first material, and a longitudinally extending monofilament, formed of a second material, fixed to the tube or embedded therein. The monofilament has a lower elongation ability than the tube. The monofilament has a tensile modulus greater than 150 Mpa, and a flexure modulus of the fluid-tight lumen is smaller than 300 Mpa.

In accordance with a further embodiment of the disclosed technology, there is provided a method of delivering a medical system into a portion of the gastrointestinal tract of a subject. The method includes delivering (e.g., orally, rectally, percutaneously and preferably orally) a delivery-state elongate tube and a fluid-tight lumen, associated with a linearizing element, into the gastrointestinal tract of the subject, such that the elongate tube is disposed at a target location within the gastrointestinal tract of the subject and a second end of the fluid-tight lumen, distal to the elongate tube, remains outside the mouth of the subject. The method further includes removing the linearizing element from the elongate tube and the fluid-tight lumen, thereby to allow the elongate tube to form the coil within the target location in the gastrointestinal tract of the subject.

In some embodiments, the medical system delivered in this manner can be used in a method for treating a subject, which additionally includes connecting the fluid-tight lumen to a negative pressure source, applying negative pressure in the range of 25-350 mmHg to the fluid tight lumen, maintaining the elongate tube within the body of the subject for a predetermined treatment duration, and following completion of the predetermined treatment duration, removing the elongate tube from the body of the subject.

In some embodiments, in which the medical system is delivered orally, the method further includes transitioning the fluid-tight lumen from the oral cavity of the subject to a nasal cavity of the subject. In some such embodiments, the removing includes removing the elongate tube via the nose of the subject.

### BRIEF DESCRIPTION OF THE FIGURES

The foregoing discussion will be understood more readily from the following detailed description when taken in conjunction with the accompanying Figures, in which:

FIG. 1A is a schematic illustration of a medical system according to embodiments of the disclosed technology, deployed in the esophagus;

FIG. 1B is a schematic illustration of an elongate tube of the medical system of FIG. 1A in a first operative state, deployed in the esophagus, when negative pressure is applied thereto, according to embodiments of the disclosed technology;

FIG. 1C is a schematic illustration of a method of testing the flexure modulus of an elongate tube or fluid tight lumen, forming part of the medical system of FIGS. 1A and 1B, according to embodiments of the disclosed technology;

FIG. 2 is a schematic sectional illustration of a fluid-tight lumen forming part of the medical system of FIGS. 1A and 1B;

FIGS. 3A and 3B are a perspective sectional illustration and a planar sectional illustration of an exemplary structure of an elongate tube forming part of the medical system of FIGS. 1A and 1B, according to embodiments of the disclosed technology;

FIGS. 4A, 4B, and 4C are, respectively, a perspective view illustration and two sectional illustrations of a segment of the elongate tube of FIGS. 3A and 3B, when in the linear delivery state, in which a relative position of the shape-forming wire and the elongate tube is suitable for forming a coil suitable for use in the medical system of FIGS. 1A and 1B, according to embodiments of the disclosed technology;

FIGS. 5A, 5B, 5C, and 5D are, respectively, a perspective view illustration and three sectional illustrations of a segment of the elongate tube of FIGS. 3A and 3B, when in the linear delivery state, in which a relative position of the shape-forming wire and the elongate tube is unsuitable for forming a coil suitable for use in the medical system of FIGS. 1A and 1B, according to embodiments of the disclosed technology;

FIGS. 6A and 6B are, respectively, a schematic perspective view illustration and a schematic sectional illustration of a coil formed from the elongate tube of FIGS. 3A and 3B, the coil being suitable for use in the medical system of FIGS. 1A and 1B;

FIGS. 7A and 7B are, respectively, a schematic perspective view illustration and a schematic sectional illustration of a coil formed from the elongate tube of FIGS. 3A and 3B, the coil being unsuitable for use in the medical system of FIGS. 1A and 1B;

FIGS. 8A, 8B, and 8C are schematic illustrations of exemplary structures of coils that can be formed of elongate tubes having a structure similar to that of the elongate tube of FIGS. 3A and 3B, which coils are suitable for use in the medical system of FIGS. 1A and 1B;

FIG. 9 is a perspective sectional illustration of an exemplary structure of an elongate tube forming part of the medical system of FIGS. 1A and 1B, according to embodiments of the disclosed technology;

FIG. 10 is a planar sectional illustration of an exemplary structure of an elongate tube forming part of the medical system of FIGS. 1A and 1B, according to embodiments of the disclosed technology;

FIGS. 11A, 11B, and 11C are, respectively, side view illustrations and an end view illustration of an exemplary structure of an elongate tube forming part of the medical system of FIGS. 1A and 1B, according to embodiments of the disclosed technology;

FIGS. 12A, 12B, and 12C are schematic illustrations of exemplary structures of a coil formed of an elongate tube forming part of the medical system of FIGS. 1A and 1B, according to embodiments of the disclosed technology;

FIG. 13 is a schematic illustration of an exemplary structure of a reinforced elongate tube forming part of the medical system of FIGS. 1A and 1B, according to embodiments of the disclosed technology;

FIG. 14 is a schematic illustration of an exemplary coil structure of an elongate tube forming part of the medical system of FIGS. 1A and 1B, according to embodiments of the disclosed technology;

FIGS. 15A, 15B, 15, and 15D are, respectively, side view illustrations, a top view illustration, and a segmented illustration an exemplary structure of an elongate tube forming part of the medical system of FIGS. 1A and 1B, according to embodiments of the disclosed technology;

FIGS. 16A, 16B, 16C, and 16D are schematic illustrations of embodiments of valves disposed at an end of an elongate tube forming part of the medical system of FIGS. 1A and 1B, according to embodiments of the disclosed technology;

FIGS. 17A, 17B, and 17C are schematic illustrations of embodiments of placement of portions of the medical system of FIGS. 1A and 1B, within the gastrointestinal tract, in order to treat a wound in the gastrointestinal tract, according to embodiments of the disclosed technology;

FIGS. 18A, 18B and 18C are schematic illustrations of steps of deploying the medical system of FIGS. 1A and 1B into the gastrointestinal tract according to an embodiment of the disclosed technology;

FIGS. 19A and 19B are schematic illustrations of steps of deploying the medical system of FIGS. 1A and 1B into the gastrointestinal tract according to an embodiment of the disclosed technology;

FIGS. 20A and 20B are schematic illustrations of steps of deploying the medical system of FIGS. 1A and 1B into the gastrointestinal tract according to an embodiment of the disclosed technology;

FIGS. 21A and 21B are schematic illustrations of a procedure of deploying a medical system, similar to that of FIGS. 1A and 1B, into the body of a subject, according to embodiments of the disclosed technology;

FIGS. 22A and 22B are schematic illustrations of steps of deploying the medical system of FIGS. 1A and 1B into the gastrointestinal tract according to an embodiment of the disclosed technology;

FIGS. 23A and 23B are schematic illustrations of anchoring the medical system of FIGS. 1A and 1B within the gastrointestinal tract according to an embodiment of the disclosed technology; and

FIGS. 24A, 24B, 24C, 24D, and 24E are schematic illustrations of steps of a procedure for maintaining the medical system of FIGS. 1A and 1B in the body of the subject via a nasal wire or tube.

### DETAILED DESCRIPTION

The principles of the medical systems and methods may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the invention is not limited in its application to the details of construction and the arrangement of the components set forth in the following description or illustrated in the drawings. The invention is capable of other implementations or of being practiced or carried out in various ways. Furthermore, it is to be understood that the phraseology and terminology employed in the disclosure is for the purpose of description and should not be regarded as limiting.

For the purposes of this application, the term “subject” relates to any mammal, particularly humans, and includes children.

In the context of the present description and claims, the terms “proximal” and “distal” are defined relative to a direction in which the system is deployed into the body of the subject. As such, an element is said to be “proximal” if it is closer to the point at which the system enters the body of the subject than other elements, and is said to be “distal” if it is further from the point at which the system enters the body of the subject than other elements.

In the context of the present description and claims, the term “wound” relates to any form of damage to the tissue, including, but not limited to, a leak, a perforation, a rupture, a tear, a cut, or a fistula in the tissue, for example in the wall of the GI tract.

In the context of the present description and claims, the term “negative pressure” relates to sub-atmospheric pressure, which may be applied, for example, to remove fluid or debris from a bodily lumen.

In the context of the present description and claims, the term “elongate tube” relates to an elongate structure having at least a portion which is tubular, i.e. at least a portion that includes an internal channel. The internal channel need not extend through the entire elongate structure, or even through a majority of the elongate structure, for the structure to be considered an “elongate tube”.

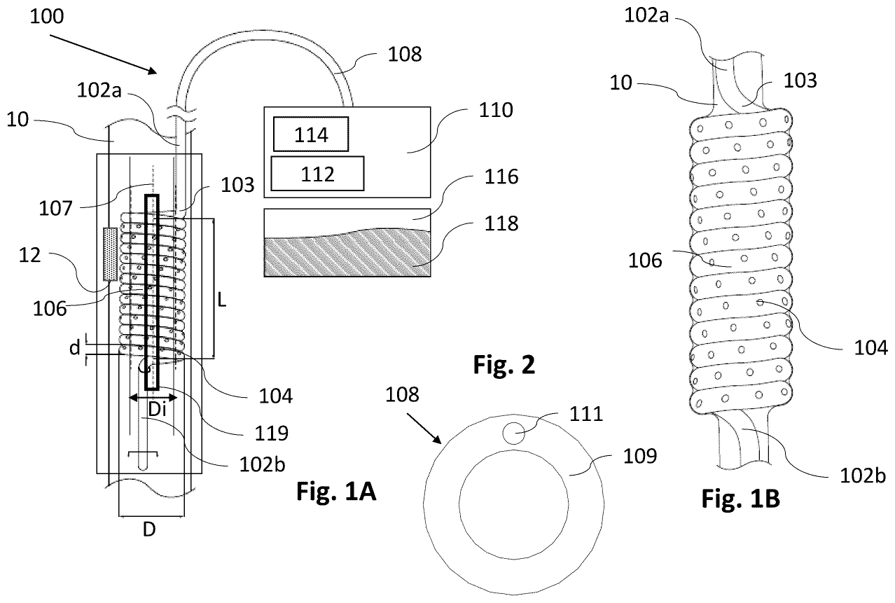

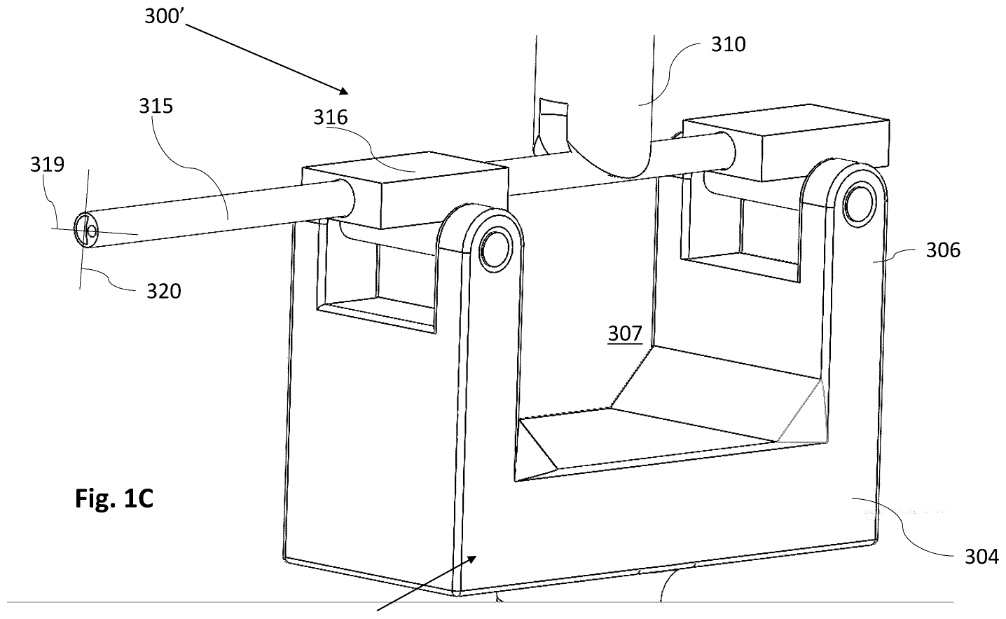

Referring now to the drawings, FIG. 1A is a schematic illustration of a medical system 100 according to embodiments of the disclosed technology, deployed in the vicinity of an endoluminal or extraluminal wound, for example in an esophagus 10 of a subject. Typically, system 100 is configured to remove fluid from the vicinity of wound 12, and/or to assist in healing thereof, by application of negative pressure to the vicinity of the deployed system. For this purpose, portions of system 100 are designed to be retained within the body of a subject for extended durations, such as longer than 24 hours, longer than 48 hours, longer than 72 hours, or even longer than a week.

System 100 includes an elongate tube 102 shaped and sized for delivery to a human esophagus, or to another portion of the human GI tract. Elongate tube 102 includes at least one channel (described in further detail hereinbelow) extending along at least a longitudinal portion of the elongate tube, and one or more portals in fluid communication with channel(s). In some embodiments, and as shown in FIG. 1A, the plurality of portals comprises a plurality of orifices 104. However, in other embodiments, the portal(s) may include one or more slots, as explained in further detail hereinbelow. For brevity, the following description relates to orifices 104, while being similarly relevant to other types of portals, such as slots. Various embodiments and characteristics of elongate tube 102 are described hereinbelow.

Elongate tube 102 has a delivery state, which is typically substantially linear, while being capable of curving to accommodate delivery into the GI tract, which is non-linear, and passing bends in the GI tract. As explained in further detail hereinbelow, in some embodiments the delivery state is only accomplished when elongate tube is 102 is associated with a linearizing element, such as a guidewire extending within the elongate tube or a tubular sheath extending around the elongate tube. In some embodiments, more than one linearizing element may be employed at different stages of delivery. For example, a tubular sheath may be employed to linearize the coil in a first stage and then a guidewire may be added as a secondary linearizing element and prior to removal of the tubular sheath. This process may ease delivery of the device via a working channel of a scope to the desired location.

Elongate tube 102 additionally has a first operative state, also termed a resting operative state, as seen in FIG. 1A, for example. The resting operative state occurs when the elongate tube is dissociated from a linearizing element, such as when the elongate tube deployed in the body of the subject, and specifically within the gastrointestinal tract. In the resting state, the elongate tube forms a coil including a plurality of loops 106, arranged around a longitudinal coil axis 107 extending through the center of the coil.

In the following description, the terms “first operative state” and “resting operative state” are used interchangeably.

A fluid-tight lumen 108 is in fluid communication with an end of elongate tube 102, typically with the channel(s) thereof. As such, the hollow of fluid-tight lumen 108 is continuous with at least one channel of elongate tube 102, or the fluid tight lumen and elongate tube share a continuous internal volume. Fluid-tight lumen 108 is adapted to couple, or couples, elongate tube 102 to a source of negative pressure (e.g., negative pressure system) 110, for delivery of negative pressure to orifices 104, via elongate tube 102 and its channel(s). Negative pressure delivered to the orifices results in removal or drainage of fluid and/or debris from the vicinity of wound 12, thus assisting in healing of the wound.

In some embodiments, and as illustrated in FIG. 1A, fluid-tight lumen 108 is in fluid communication with a proximal end 102a of elongate tube 102. Additionally, the fluid-tight lumen 108 is adapted to couple to a source of negative pressure 110, and to deliver negative pressure to the elongate tube 102 via the proximal end 102a. However, depending on the direction of deployment, fluid-tight lumen 108 may alternately be coupled to a distal end of the elongate tube, provided that it is coupled to source of negative pressure 110. In some embodiments, the system includes the source of negative pressure 110, for example in the form of a vacuum generator, which may be fixed or portable. In other embodiments, the system does not include the source of negative pressure, and merely interacts or is adapted to connect with the source of negative pressure such as a vacuum system.

In some embodiments, source of negative pressure 110 includes a controller 112 adapted to regulate the negative pressure provided by source 110, within a predefined pressure range, to remove fluid at least from a vicinity of the extraluminal or endoluminal wound, a portion of the internal surface of esophagus 10, or of the gastrointestinal tract. For example, controller 112 may be adapted to regulate the negative pressure for removal of fluid from an area of esophagus 10 including the extraluminal or endoluminal wound.

In some embodiments, source of negative pressure 110 may further include, or be associated with, least one sensor 114 adapted to sense at least one characteristic of the fluid removed from the gastrointestinal tract such as pressure or flow rate. Sensor(s) 114 is functionally associated with controller 112, such that the controller is adapted to adjust one or more operating parameters of the source of negative pressure 110 in response to input received from the sensor(s), which input relates to the at least one characteristic of the fluid. Sensor(s) 114 may be positioned in proximity to controller 112, or in proximity to elongate tube 102.

In some embodiments, or at certain times, elongate tube 102 may be dissociated from the source of negative pressure 110, and may be coupled instead to a source of fluid, indicated by reference numeral 116 in FIG. 1A. Source 116 includes fluid 118, which may be a flushing fluid or a treatment fluid. In some embodiments, fluid 118 may be supplied, via the channel(s) and orifices 104, into the gastrointestinal tract. For example, the fluid may be supplied to dislodge debris caught in the orifices or in the channel. As another example, the fluid may include an irrigation or cleansing fluid, a medicament (e.g., anti-inflammatory agent), and/or an antimicrobial (e.g., antibiotic or antibacterial), to assist in healing of wound 12.

In some embodiments, the fluid may be a flushing fluid. In some embodiments, the fluid may be a medicament fluid, such as an antimicrobial fluid or a tissue-growth promoting fluid. In some embodiments, the fluid may be a contrast fluid. In some embodiments, the fluid may be ionized gas. In some embodiments, the fluid may be carbon dioxide. In some embodiments, the fluid may be a fluid configured to modify a characteristic of the coil, such as a low temperature fluid.

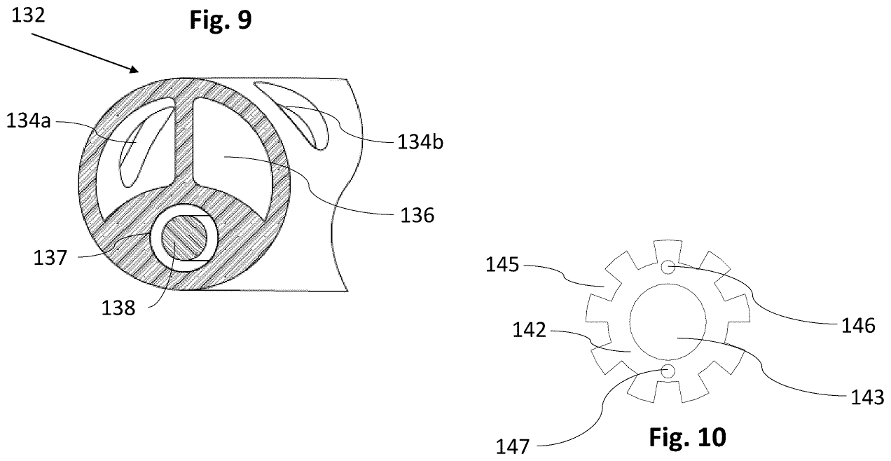

In other embodiments, the source of fluid 116 as well as the source of negative pressure 110 may both be connected to elongate tube 102. In some such embodiments, the elongate tube may include multiple channels, as explained in further detail hereinbelow, for example with respect to FIG. 9.

In some embodiments, after elongate tube 102 has formed the coil, an additional tube 119 may be pushed into the center of the coil, to extend therethrough. For example, in some embodiments, additional tube 119 may be an internal support tube, supporting the structure of the coil from within. As another example, in some embodiments, additional tube 119 may be a feeding tube, adapted for delivery of food to the stomach of the subject, via the coil in the esophagus.

It is a particular feature of the disclosed technology that additional tube 119 may extend through the coil even during application of negative pressure to the coil for treatment using the system of the disclosed technology, as disclosed herein.

In some embodiments, one or more of sensors 114 may be associated with the distal end of the coil, the central volume of the coil, the exterior surface of the coil, or the internal tube 119. The sensor may be any sensor suitable for assisting in the treatment process described herein. For example, the sensor may include an image capturing sensor, such as a stills or video camera, adapted to capture images providing information regarding the positioning of the elongate tube within the gastrointestinal tract. As another example, the sensor may be a pressure sensor adapted to provide information about a pressure applied to, or applied within, the elongate tube.

For use of system 100, elongate tube 102 is delivered into the gastrointestinal tract of the subject, together with a linearizing element causing the elongate tube to be in the linearized delivery state, and with a distal portion of fluid-tight lumen 108, which is attached to elongate tube 102. Once within the gastrointestinal tract, the linearizing element is removed from elongate tube 102, and the elongate tube reverts to its resting operative state, by forming coil 106 within the lumen of the gastrointestinal tract. In some embodiments, the coil is sized and configured to establish contact with the inner wall of the GI tract and/or to have a geometric anisotropy.

Elongate tube 102 is configured to form the coil while it is disposed within the lumen of the GI tract, or within a bodily lumen having a diameter smaller than 9 cm, smaller than 5 cm, or smaller than 3.5 cm. In some embodiments, elongate tube 102 is configured to form a coil while it is disposed within the esophagus of the subject.

Once the coil has been formed within the gastrointestinal tract, negative pressure is delivered to orifices 104 of coil 106 from negative pressure source 110 via fluid tight lumen 108 and the channel(s) of elongate tube 102, for removal or drainage of fluid and/or debris from the vicinity of wound 12.

As seen in FIG. 1B, depending on the degree of negative pressure applied to coil 106 disposed in the lumen of the gastrointestinal tract, the portion of the gastrointestinal tract in which the coil is located, such as esophagus 10, collapses about the coil, thus creating a negative pressure chamber, or multiple negative pressure regions, within the gastrointestinal tract. As such, coil 106 formed by elongate tube 102 must be sufficiently strong to retain a, possibly modified, coiled shape when negative pressure is applied thereto, when the portion of the gastrointestinal tract collapses thereon, and during normal operation (e.g., application of peristaltic pressure) of the gastrointestinal tract.

Specifically, in order to enable the coiling and linearizing of elongate tube 102, the elongate tube must be flexible in a radial direction—the direction of rotation of the coil. By contrast, the elongate tube 102 must be significantly less flexible in other directions, and specifically in the axial direction of coil axis 107, in order to retain the coiled structure and prevent elongation of coil 106, or significant changes to the pitch between the loops of the coil, even under negative pressure applied to the coil and under external pressures applied within the gastrointestinal tract. For example, the elongate tube must be rigid enough to retain its coil shape, and to prevent the coil from excessive elongation, during peristaltic motion in the gastrointestinal tract.

For example, when a coil 106 formed of the elongate tube 102 is deployed within the gastrointestinal tract of a landrace female swine having a weight in the range of 60-90 kg, mechanical characteristics of the coil formed by the elongate tube prevent total coil elongation greater than 100%, greater than 75%, or greater than 50% when retained in the gastrointestinal tract for at least 48 hours, under natural peristaltic forces within the gastrointestinal tract. In some embodiments, the total elongation of the coil is restrained as discussed herein when measured when negative pressure is applied to the coil. In some embodiments, the total elongation of the coil is restrained as discussed herein when measured without negative pressure being applied to the coil.

In order to form coil 106 from elongate tube 102, and retail the shape of the coil within the gastrointestinal tract, the inventors have found that elongate tube 102 must have a first flexure modulus in a coil-radial direction of the elongate tube, and a second flexure modulus in a coil-axial direction of the elongate tube, where the second flexure modulus is greater than the first flexure modulus. The first flexure modulus and second flexure modulus of elongate tube 102 are typically measured in accordance with a deflection test, for example as defined in ASTM D790, on a linear segment of elongate tube 102.

Elongate tube 102 is typically geometrically anisotropic, such that it has distinct flexure modulus characteristics on different axes thereof. Therefore, it is important that the elongate tube does not rotate during the measurement of the flexure modulus, and it may be desirable to ensure that the orientation of a sample of the elongate tube is retained during measurement, so that the measurement will be obtained in the desired direction. As such, the flexure modulus of elongate tube 102, in each of the coil-radial and coil-axial directions, may be measured using a device 300, shown in FIG. 1C.

In the illustrated example, the flexure modulus of elongate tube 102 is measured on a sample 315 of the tube, prior to the elongate tube it being subjected to treatment for the tube to coil. Sample 315 has the same mechanical structure and composition as elongate tube 102, other than the coiling aspect.

Device 300, shown in FIG. 1C, includes a substantially U-shaped jig 302 having a base 304 and arms 306 disposed at predetermined distance from each other, such that a cavity 307 is formed between the arms. Device 300 further includes an upper portion 308 including a pusher 310, disposed above a center point between arms 306.

Device 300 further includes blocks 316, each having a channel 318 extending therethrough, which rest above the upper end of arms 306 of the jig. Channels 318 of blocks 316 are sized and dimensioned to hold sample 315 snugly, so that the sample cannot rotate relative to blocks 316 within the blocks. Blocks 316 merely rest on upper ends of arms 306 and have freedom of motion, along their longitudinal axis, relative to the arms.

In use, sample 315 is placed within channels 318 of blocks 316 disposed on jig 302, such that the sample, together with blocks 316, is not held or anchored to the jig or to another element outside of the jig. Subsequently, pusher 310 is lowered onto the sample, pushing the sample substantially vertically into cavity 307 until the sample reaches a predetermined deflection distance d₁, and the force used for that deflection is then measured, in accordance with the Standard requirements.

A first flexure modulus of sample 315 is measured along axis 319 which reflects the coil-radial direction. A second flexure modulus of sample 315 is measured along axis 320, which is substantially perpendicular to axis 319 and reflects the coil-axial direction.

As described hereinabove, the second flexure modulus in the coil-axial direction is greater than the first flexure modulus in the coil-radial direction.

In some embodiments, the second flexure modulus in the coil-axial direction is at least twice as large as the first flexure modulus in the coil-radial direction.

The relationship between the second flexure modulus in the coil-axial direction and the first flexure modulus in the coil radial direction may be reflected as a dimensionless ratio. In some embodiments, the dimensionless ratio is greater than 1:1, greater than 2:1, greater than 3:1, or greater than 4:1.

In some embodiments, the first flexure modulus in the coil radial direction is in the range of 20 Mpa to 3000 Mpa, or in the range of 20 Mpa to 1000 Mpa.

In some embodiments, the second flexure modulus in the coil-axial direction is greater than 500 Mpa.

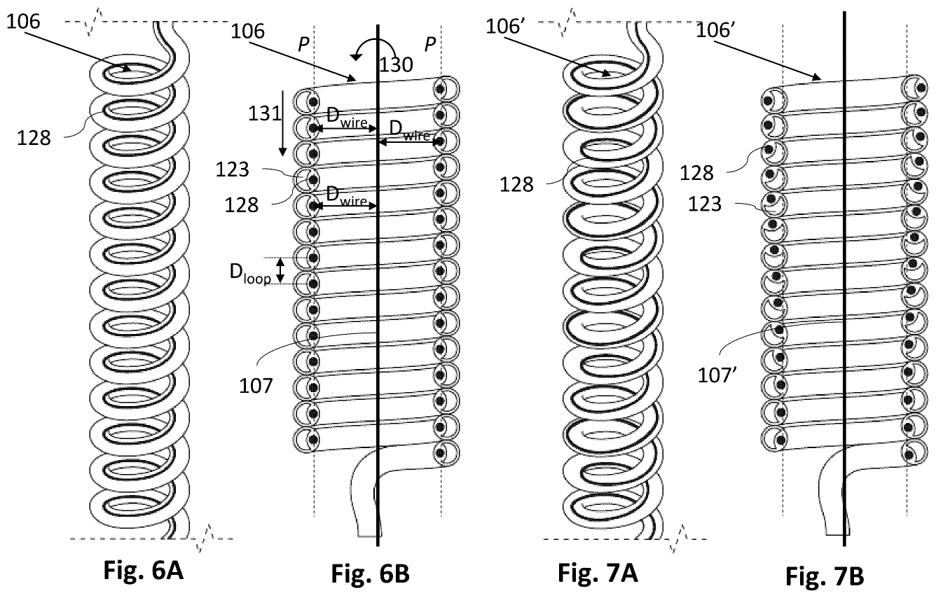

In some embodiments, for a cross section of elongate tube 102, a radial moment of inertia of the elongate tube, measured along axis 319 in FIG. 1C, is smaller than an axial moment of inertia of the elongate tube, measured along axis 320 in FIG. 1C. In some embodiments, both the radial moment of inertia and the axial moment of inertia of the elongate tube are in the range of 15-30 mm+. The coil-radial and coil-axial directions of coil 106 formed of elongate tube 102 are also indicated by respective arrows 130 and 131, in FIG. 6B.

While coil 106 is disposed within the gastrointestinal tract, fluid-tight lumen 108 extends from within the body of the subject to the exterior of the body of the subject, typically passing through the nasal cavity, as explained in further detail hereinbelow. As such, it is very important that the fluid-tight lumen be sufficiently flexible to pass through the nasal cavity with minimal discomfort to the subject. At the same time, fluid-tight lumen 108 must be sufficiently resistant to elongation, to allow association thereof with the linearizing element, and delivery of elongate tube 102 and of the distal portion of the fluid tight lumen into the body of the subject. Mechanical properties of fluid-tight lumen 108 which provide and/or meet these requirements, are described in further detail hereinbelow, with reference to FIG. 2.

In some embodiments, upon completion of treatment, the entirety of elongate tube 102, as well as the fluid-tight lumen 108 are removed from the body of the subject.

In some embodiments, at least a portion of elongate tube 102, or the entirety of elongate tube 102, may be detachable from fluid-tight lumen 108. In some embodiments, the detachable portion (or entirety) of elongate tube 102 is formed of material which may be naturally excreted from the body of the subject following detachment from fluid tight lumen 108. In other embodiments, the detachable portion (or entirety) of elongate tube 102 may be formed of a biodegradable material, and may be degraded or decomposed, within the body of the subject, following detachment from fluid tight lumen 108.

The following description relates to some additional characteristics of various components of system 100, as illustrated in FIGS. 1A and 1B.

As seen in FIGS. 1A and 1B, it is desirable that coil 106 be disposed within the lumen of the GI tract, such that coil axis 107 is parallel to, or at least not too tilted with respect to, a lumen-longitudinal-axis of the lumen of the gastrointestinal tract (e.g., esophagus 10), and that no kinks or crimps are formed in the elongate tube during coiling. In some embodiments, elongate tube 102 coils within the GI tract such that an angle between the lumen-longitudinal-axis and the coil-axis is not greater than 30 degrees, not greater than 25 degrees, not greater than 20 degrees, not greater than 15 degrees, not greater than 10 degrees, or not greater than 5 degrees. As explained herein, structural aspects of components of system 100 assist in maintaining the correct orientation of the coil.

In some embodiments, in the coiled state of elongate tube 102, a lead extending from distal end section 102b of the coiled tube remains linear and does not coil. In some embodiments, a length of distal end section 102b of the coiled tube is greater than an external diameter of the coil. In some embodiments, in the coiled state of elongate tube 102, a proximal end section 102a of the coiled tube, has a different longitudinal direction than the direction of rotation of the coil, such that a shoulder 103 is formed. In some other embodiments, shoulder 103 may be formed by a distal end of fluid-tight lumen 108 having a different longitudinal direction than a more proximal portion of the fluid-tight lumen. In some embodiments, shoulder 103 may be in the range of 0-60 degrees offset from the longitudinal axis of the coil.

In some embodiments, the linear lead extending from distal end section 102b and/or shoulder 103 of the coil assist in ensuring that when the elongate tube 102 coils within the lumen of the gastrointestinal tract, the desired angular relationship between coil axis 107 and the lumen-longitudinal-axis is achieved.

In some embodiments, at least a portion of elongate tube 102, or the entirety thereof, is further covered by an additional layer of material such as a netting configured to add friction to the surface of the elongate tube. In some embodiments, the entirety elongate tube 102, in the coiled state, is further covered or contained by an additional layer of material such as a netting. In some embodiments, the coil may move freely within the material covering and/or the material covering may be formed of an elastic material.

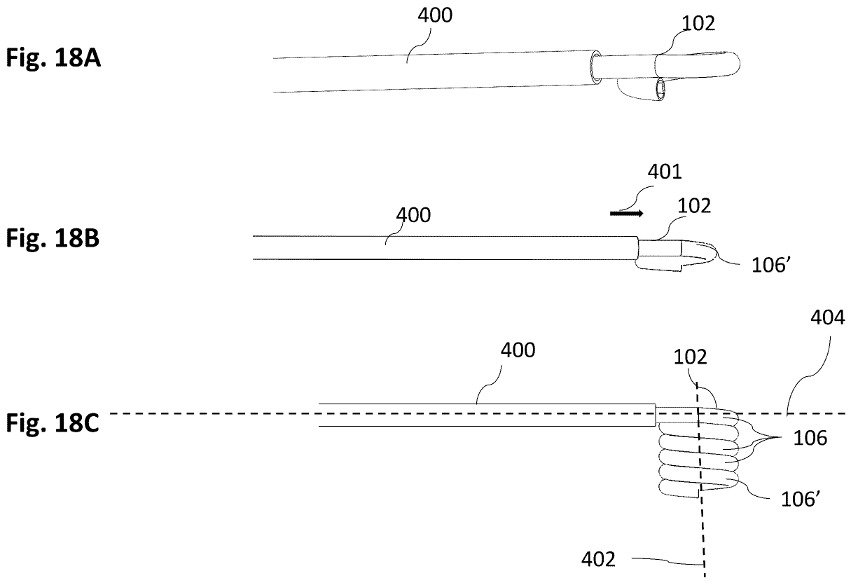

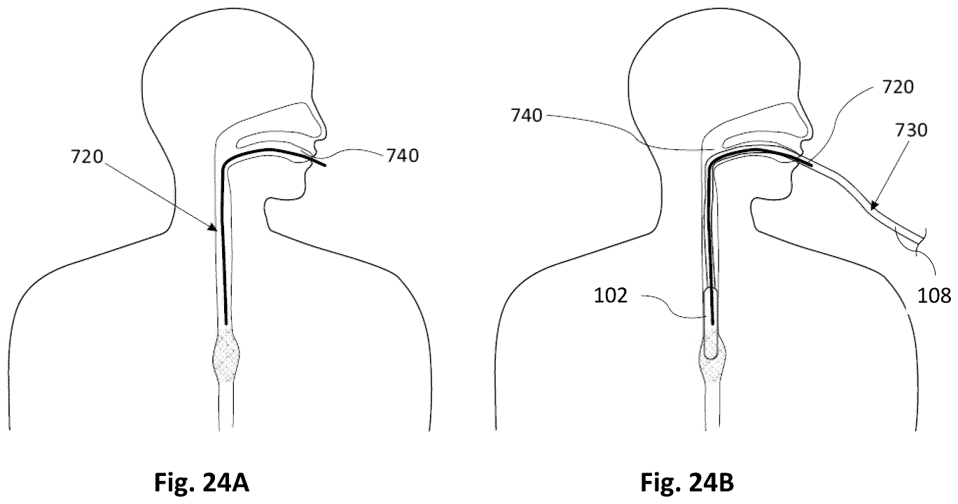

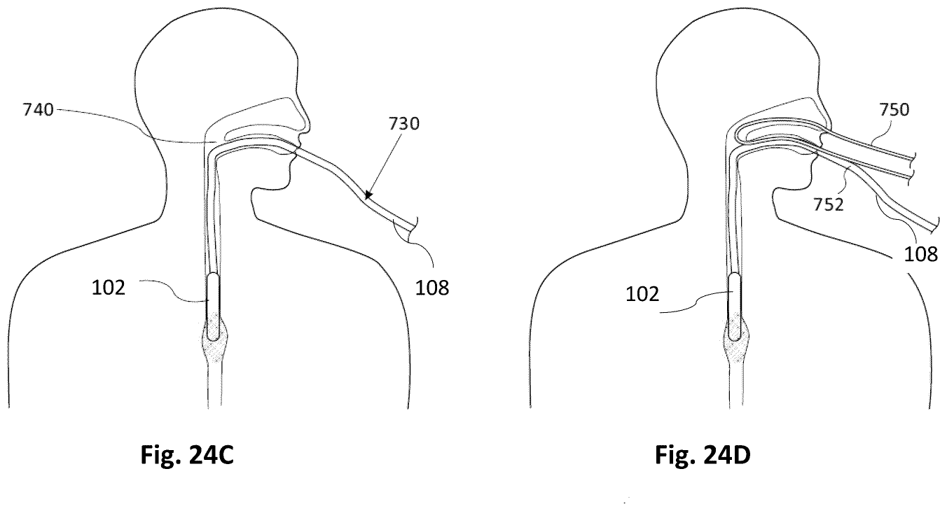

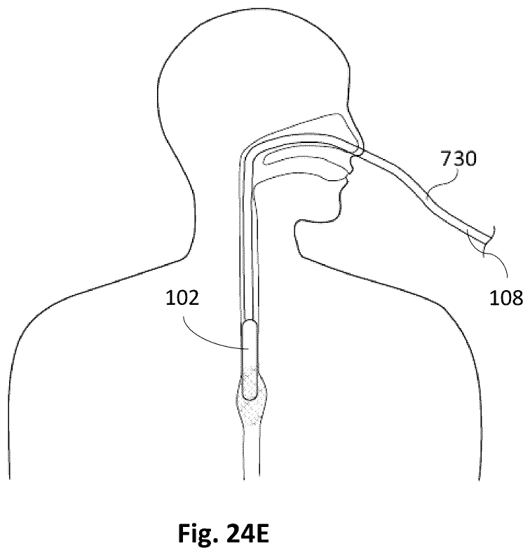

Reference is now additionally made to FIG. 2, which is a cross sectional illustration of an embodiment of fluid-tight lumen 108. As mentioned above, it is a particular feature of the disclosed technology that fluid tight lumen 108 must be sufficiently flexible to extend through the nasal cavity of the subject, as described hereinbelow with respect to FIGS. 24A to 24E. At the same time, fluid tight lumen 108 must also resist elongation force is applied thereto, in order to allow introduction of the fluid tight lumen into the body of the subject within a sheath or over-tube, or using a guidewire extending through the fluid tight lumen, as explained hereinbelow with respect to FIGS. 18A to 21B.

To achieve these seemingly contradictory goals, and as seen in FIG. 2, in some embodiments, fluid tight lumen 108 includes a tube 109, having a longitudinal monofilament 111 embedded therein or fixed thereto.

It is to be appreciated that, within the context of the present application and claims, tube 109 is considered to be a tube even if its tubular structure requires inclusion of monofilament 111, and upon removal of monofilament 111, the material of tube 109 forms a C-shape, and not a closed tube.

Tube 109 and monofilament 111 are formed of different materials, wherein the context of the present application and claims, the term “different materials” is defined as materials having different mechanical properties. The different materials may have distinct structural properties (e.g. be completely different materials, such as a rubber and a metal), or may have similar structural properties, but distinct mechanical properties).

Specifically, tube 109 and monofilament 111 have distinct mechanical properties so as to ensure that the fluid-tight lumen has a sufficiently high tensile modulus so as to resist elongation during delivery into the body of the subject, while at the same time having a sufficiently low flexure modulus so as to reduce discomfort to the subject when the fluid tight lumen is disposed in the nasal cavity of the subject for an extended duration.

In some embodiments, tube 109 and monofilament 111 are formed of different materials having distinct structural properties, or distinct structures. For example, tube 109 may be formed of silicone or polyurethane, and monofilament 111 may be formed of a metal, such as nitinol. In some embodiments, monofilament 111 may formed of a polymer, such as polyether-ether-ketone (PEEK), polyethylene (PE), polyethylene terephthalate (PET), or polyether block amide (PEBAX). In some embodiments, monofilament 111 is non-absorbable in a human GI tract.

In some other embodiments, tube 109 and monofilament 111 may be formed of two materials having similar chemical or structural properties, but distinct mechanical properties. For example, both tube 109 and monofilament 111 may be formed of silicone or polyurethane, having different durometer measures, different flexure moduli, or different tensile moduli.

In some embodiments, tube 109 has a substantially round cross-section.

Monofilament 111 may have any suitable cross section in a direction perpendicular to the longitudinal axis of the monofilament, including a circular cross section, an oval cross section, or a polygonal cross section. In some embodiments, monofilament 111 may comprise a flat strip.

In some embodiments, a greatest dimension of the cross-section of monofilament 111 is in the range of 0.1 mm to 5.0 mm, 0.1 mm to 1.5 mm, in the range of 0.1 mm to 1.0 mm, in the range of 0.1 mm to 0.8 mm, or in the range of 0.1 mm to 0.5 mm.

It is a particular feature of the disclosed technology that tube 109 is substantially flexible, and provides the required flexure modulus of fluid-tight lumen 108, while monofilament 111 has a higher tensile modulus than that of tube 109, and assists in ensuring that fluid-tight lumen 108 have a suitable tensile modulus, and be sufficiently resistant to elongation.

As such, in some embodiments, monofilament 111 has a tensile modulus greater than 60 Mpa, greater than 100 Mpa, greater than 150 Mpa, greater than 200 Mpa, greater than 300 Mpa, greater than 400 Mpa, or greater than 500 Mpa.

In some embodiments, and in order to be able to sit within the nasal cavity without causing the subject too much discomfort, fluid-tight lumen 108 has a minimal bending radius of 15 cm or more, without forming kinks in the fluid-tight lumen.

For the same reason, in some embodiments, fluid-tight lumen 108 has a flexure modulus smaller than 1500 Mpa, smaller than 1000 Mpa, smaller than 500 Mpa, smaller than 300 Mpa, smaller than 200 Mpa, or smaller than 100 Mpa, despite the presence therein of monofilament 111. The flexure modulus of fluid-tight lumen 108 is typically measured in accordance with a deflection test, for example as defined in ASTM D790, using any means known in the art. In some embodiments, the flexure modulus of fluid-tight lumen 108 may be measured using the device described hereinabove with respect to FIG. 1C.

In some embodiments, when a force of 10N is applied axially to fluid-tight lumen 108, an elongation percentage of the fluid-tight lumen is not greater than 5%, not greater than 2%, or not greater than 1%.

In some embodiments, when a force of 15N is applied axially to fluid-tight lumen 108, an elongation percentage of the fluid-tight lumen, is not greater than 5% or not greater than 2%. In some embodiments, when a force of 20N is applied axially to fluid-tight lumen 108, an elongation percentage of the fluid-tight lumen, is not greater than 5% or not greater than 2%.

In some embodiments, when a force of 25N is applied axially to fluid-tight lumen 108, an elongation percentage of the fluid-tight lumen, is not greater than 5% or not greater than 2%.

In some embodiments, when a force of 30N is applied axially to fluid-tight lumen 108, an elongation percentage of the fluid-tight lumen, is not greater than 5% or not greater than 2%.

Testing of the elongation percentage, or the resistance of fluid-tight lumen 108 to elongation, may be carried out using methods known in the art. For example, a sample of fluid-tight lumen 108 having a predetermined length may be held at opposing ends, and pulled in opposing directions, thereby applying an axial force of a known quantity. An under-force length of the sample during application of the axial force is compared to the predetermined length, to evaluate a degree of elongation of the sample.

The resistance of fluid-tight lumen 108 to elongation can also be measured in terms of the tensile modulus of the fluid-tight lumen. In some embodiments, the tensile modulus of fluid-tight lumen 108 is at least 100 Mpa, at least 200 Mpa, at least 300 Mpa, at least 400 Mpa, or at least 500 Mpa.

In some embodiments, the tensile modulus of fluid-tight lumen 108 is greater than the flexure modulus of the fluid-tight lumen.

In some embodiments, the tensile modulus of fluid-tight lumen 108 is twice as large as the flexure modulus of the fluid-tight lumen.

In some embodiments, for fluid-tight lumen 108, the ratio between the tensile modulus and the flexure modulus is greater than 1:1, greater than 2:1, greater than 3:1, or greater than 4:1.

In some embodiments, the flexure modulus of fluid-tight lumen 108 is substantially equal to the flexure modulus of tube 109, while the tensile-modulus of the fluid-tight lumen is greater, and in some embodiments at least twice as large, as the tensile modulus of tube 109. In some embodiments, the tensile modulus of fluid-tight lumen 108 is substantially equal to the tensile modulus of monofilament 111.

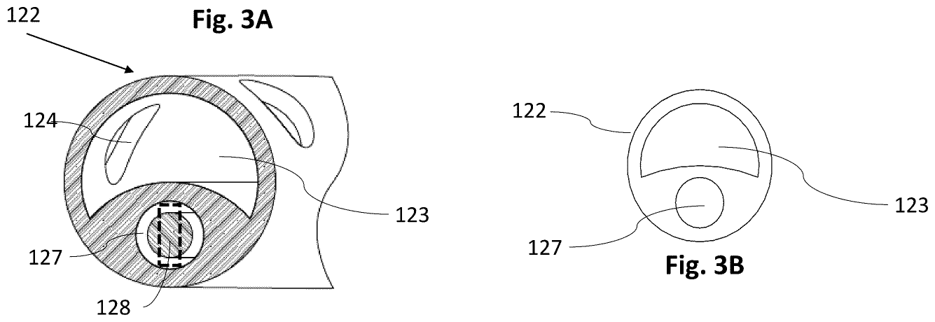

Reference is now additionally made to FIGS. 3A and 3B, which are a perspective sectional illustration and a planar sectional illustration of an exemplary structure of an elongate tube 122, similar to elongate tube 102 of FIGS. 1A and 1B, according to embodiments of the disclosed technology

In the embodiment illustrated in FIGS. 3A and 3B, elongate tube 122 includes a main channel 123, and orifices 124. Channel 123 is in fluid communication with source 110 of negative pressure and with orifices 124, and functions as a vacuum-delivery channel adapted to deliver negative pressure from the source of negative pressure to the orifices. Channel 123 may have any suitable cross-sectional shape or area, including a crescent cross-sectional shape as illustrated, a circular cross-sectional shape, an oval cross-sectional shape, and the like. In the embodiment of FIGS. 3A and 3B, channel 123 may extend longitudinally along the entire length of elongate tube 122, or along a portion of the tube.

In some embodiments, and as illustrated, elongate tube 122 may have a round cross section, in a direction perpendicular to its longitudinal axis.

In some embodiments, elongate tube 122 may comprise, or may consist of, a radiopaque marker, radioactive marker, magnetic marker, and/or magnetic resonance marker. In some embodiments, elongate tube 122 may comprise, or may consist of, a metal, a natural or elastic polymer, a plastic, a shape memory alloy, and/or a super elastic alloy, a biodegradable material, a bioresorbable material, and/or a bioabsorbable material.

In some embodiments, elongate tube 122 may comprise, or may consist of, a shape memory, elastic or super-elastic material adapted to form the coil in the resting operative state. For example, in some embodiments, elongate tube 122 may be formed of the shape memory, elastic, or super-elastic material.

In some embodiments, the cross-sectional area of the orifices 124 increases along the length of the elongate tube, or of the coil, from the proximal end towards the distal end. In some such embodiments, the cross-sectional area of the distal-most orifice is at least 50% greater than the cross-sectional area of the proximal-most orifice.

In some embodiments, elongate tube 122 may further include a wire-accommodating channel 127, adapted to fixedly accommodate a shape-forming wire 128. Shape-forming wire 128 is configured for directing formation of the coil when the elongate tube is dissociated from a linearizing element, as described in further detail hereinbelow, or when the elongate tube is in the resting operative state.

As used herein, shape-forming wire 128 may be a wire or monofilament. Shape-forming wire 128 may have any suitable cross section, including a circular cross section, an oval cross section, or a polygonal cross section. In some embodiments, shape-forming wire 128 may comprise a flat strip. In the illustrated embodiment, shape-forming wire 128 has a circular cross section, while an alternative possible shape-forming wire 128a, having a rectangular cross section, is indicated in dashed lines in FIG. 3A.

In some embodiments, shape-forming wire 128 may be embedded in the material of elongate tube 122. In such embodiments, wire-accommodating channel 127 would be obviated.

As suggested by its name, shape-forming wire 128 is configured for direction formation of coil 106. In order to turn elongate tube 122 into a consistent coil shape within the lumen of the GI tract, and for that coil shape to have suitable mechanical characteristics for being disposed within and delivering negative pressure to the gastrointestinal tract for an extended duration, shape-forming wire must meet several structural and mechanical requirements.

For example, shape-forming wire 128 has an elastic range greater than 0.5%, in order to accommodate formation of the coil.

In some embodiments, shape-forming wire 128 may have a lower yield strain than elongate tube 122. Additionally, elongate tube 122 may be coextruded with shape-forming wire 128, for example from two polymeric materials. For example, shape-forming wire 128 may be formed of a material having a higher Young's modulus value than the material of the elongate tube 122. Such selection of materials is enabled by the yield strain on shape-forming wire 128 being low. In some embodiments, both the elongate tube 122 and shape-forming wire 128 are formed of thermoplastic materials having a thermoforming temperature to allow plastic deformation to form a coil shape for example in the range of 80-150 degrees Celsius, and a melting point above the thermoforming temperature.

In some embodiments, elongate tube 122 including shape-forming wire 128 have Young's modulus E that meets the following equation, where I is the second moment of inertia:

$${E}_{wire} * {I}_{wire} \geq \frac{{E}_{Elongate Tube} * {I}_{Elongate Tube}}{2}$$

In some embodiments, shape-forming wire 128 may comprise, or be formed of, a shape-memory material or a super elastic material. In some embodiments, shape-forming wire 128 may comprise, or may be formed of, a spring alloy, such as nitinol. In some embodiments, the material of shape-forming wire 128 has critical yield strain or elastic strain of at least 0.5%, and in some embodiments more than 3% (0.03). In some embodiments, the material of shape-forming wire 128 has a Young's modulus of at least 50 Mpa.

In some embodiments, shape-forming wire 128 is not degradable in a human GI tract. In some embodiments, shape-forming wire 128 is not absorbable in a human GI tract.

In some embodiments, shape-forming wire 128 may have a circular cross section having diameter d_{w} (shown in FIG. 3A), or a non-circular cross section having a greatest cross-sectional length d_{g} and a smallest cross-sectional length dₛ (both shown with respect to alternative shape-forming wire 128a in FIG. 3A). In some embodiments, dimension d_{w} or d_{g} is smaller than 2.0 mm, 1.5 mm, 1.2 mm, 1.0 mm, or 0.8 mm. In some embodiments, dimension d_{w} or dₛ is greater than 0.1 mm, greater than 0.2 mm, greater than 0.3 mm, greater than 0.4 mm, greater than 0.5 mm, or greater than 0.7 mm.

In some embodiments, any cross-sectional dimension of shape-forming wire 128 is in the range of 0.1 mm to 1.5 mm, 0.2 mm to 1.5 mm, 0.3 mm to 1.5 mm, 0.4 mm to 1.5 mm, 0.4 mm to 1.2 mm, 0.4 mm to 1.0 mm, 0.5 mm to 1.0 mm, or 0.7 mm to 1.0 mm, 0.3 mm to 0.7 mm, 0.4 mm to 0.7 mm, or 0.5 mm to 0.7 mm.

In order for shape-forming wire 128 to correctly drive formation of a coil from elongate tube 122, and for the resulting coil to have suitable mechanical properties as described further hereinbelow, the rotational orientation of shape-forming wire 128, relative to elongate tube 122, must remain fixed throughout the entire length of the shape-forming wire.

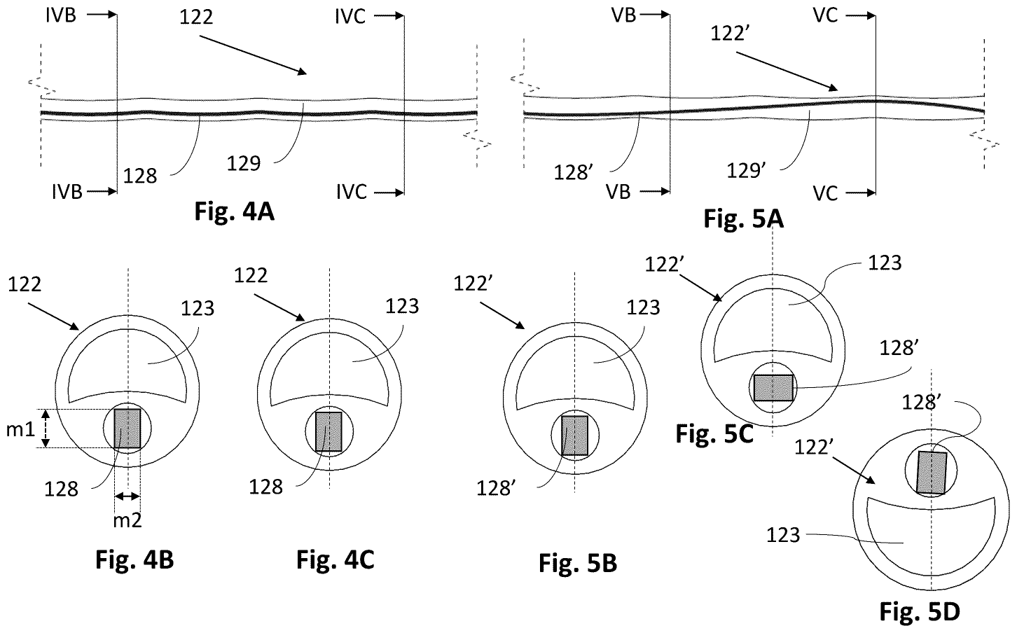

This aspect is demonstrated by additional reference to FIGS. 4A, 4B, and 4C, which show a segment 129 of the elongate tube 122, when in the linear delivery state, in which the relative rotational orientation of shape-forming wire 128 and elongate tube 122 is fixed in a desired orientation, and to FIGS. 5A, 5B, and 5C, which show a segment 129′ of an elongate tube 122′, when in the linear delivery state, in which the relative rotational orientation of shape-forming wire 128′ and elongate tube 122′ is not fixed. As such, elongate tube 122′ would not be suitable for forming a coil 106 having the required properties for use as part of system 100, as explained in further detail herein. For the illustration, shape-forming wires 128 and 128′ a cross-section of shape-forming wires 128 and 128′, in a direction perpendicular to longitudinal axes thereof, is rectangular, and has a larger aspect indicated by m1 and a smaller aspect indicated by m2, as seen in FIG. 4B.

FIGS. 4B and 4C are sectional illustrations of two portions of linearized segment 129 taken along section lines IVB-IVB and IVC-IVC, respectively. Similarly, FIGS. 5B and 5C are sectional illustrations of two portions of linearized segment 129′ taken along section lines VB-VB and VC-VC, respectively.

As seen in FIG. 4A, in linearized segment 129 of elongate tube 122, the position of shape-forming wire 128 remains the same, here shows as being disposed toward the lower side of the elongate tube, through the entire length of the segment. As seen from comparison of FIGS. 4B and 4C, throughout the entire length of segment 129, elongate tube 128 is arranged such that larger aspect m1 of shape-forming wire 128 is substantially aligned with a height of channel 123 of the tube and with a diameter of the tube, marked by a dashed line.

By contrast, in FIG. 5A, the position of shape-forming wire 128′ relative to elongate tube 122′ changes along the longitudinal length of linearized segment 129′. For example, in the left side of FIG. 5A, shape-forming wire 128′ is disposed near the lower side of elongate tube 122′, whereas in the right side of FIG. 5A, the shape-forming wire is disposed near the upper side of the elongate tube.

This change in the positioning of shape-forming wire 128′ within elongate tube 122′ can result from rotation of the shape-forming wire relative to the elongate tube, as evident from comparison of FIGS. 5B and 5C, and/or from rotation of elongate tube 122′, or of linearized segment 129′, about its longitudinal axis, as evident from comparison of FIGS. 5B and 5D. Specifically, in FIG. 5B, larger aspect m1 of shape-forming wire 128′ is substantially perpendicular to the height of channel 123′ of elongate tube 122′ and to the diameter of the elongate tube, marked by a dashed line. Additionally, shape-forming wire 128′ is disposed at the lower side of segment 129′, and channel 123 is disposed above the shape-forming wire.

However, in FIG. 5C, shape-forming wire 128′ is rotated by 90 degrees relative to elongate tube 122′ in comparison to its position in FIG. 5B, such that largest aspect m1 of the shape-forming wire is now aligned with the height of channel 123 and with the diameter of the tube.

In FIG. 5D, the entire segment 129′ has rotated about its longitudinal axis, such that shape-forming wire 128′ is disposed at the upper side of segment 129′, and channel 123 is disposed beneath the shape forming wire.

The Inventors have discovered that such rotation of the shape-forming wire relative to the elongate tube (e.g., within its accommodating channel) and/or rotation of the elongate tube about its longitudinal axis is detrimental to formation of a coil 106 within the lumen to the gastrointestinal tract, and/or to the ability of a coil formed from such an elongate tube to retain its position and function, within the gastrointestinal tract, during application of negative pressure to the gastrointestinal tract and/or during peristaltic motion within the gastrointestinal tract. Additionally, the inventors have found that rotation of the shape-forming wire relative to the elongate tube and/or rotation of the elongate tube about its longitudinal axis results in improper linearization of the elongate tube, e.g., upon introduction of a guidewire thereinto as explained hereinbelow. Such improper linearization can cause difficulty in passing the linearized elongate tube through the working channel of a delivery tool such as an endoscope, and can lead to kinking of the coil formed within the gastrointestinal tract.

As such, the Inventors have discovered that the fixed rotational orientation between the shape-forming wire and the elongate tube, and lack of rotation of the elongate tube about its own longitudinal axis, is critical to functionality of elongate tube 122, and to functionality of system 100.

In addition to the rotational orientation between the elongate tube and the shape-forming wire being fixed, in order to form coil 106 shown in FIGS. 1A and 1B, it is also important that the elongate tube not rotate about its own longitudinal axis during coiling thereof.

FIGS. 6A and 6B are, respectively, a schematic perspective view illustration and a schematic sectional illustration of a coil 106 formed from the elongate tube 122, the coil being suitable for use in the medical system 100.

As seen in FIGS. 6A and 6B, in coil 106, shape-forming wire 128 retains its location, relative to elongate tube 122, throughout all portions of the coil. Stated differently, throughout the coil, elongate tube 122 does not rotate about its own longitudinal axis, and thus the relative position of channel 123 and of shape-forming wire 128 remain substantially the same throughout the coil, and in each cross section of the elongate tube in a cross section of the coil as shown in FIG. 6B.

As a result, as seen in FIG. 6A, shape-forming wire 128 is always disposed at, or adjacent, an upper portion of each loop of the coil, and does not shift to other positions within the coil.

In the illustration shown in FIG. 6B, in each loop of coil 106, shape-forming wire 128 is disposed at, or adjacent, an interior circumference of coil 106 (in both sections thereof), while channel 123 is disposed at, or adjacent, an exterior circumference of the coil. As such, in the sections of elongate tube 122 on the left-hand side of FIG. 6B and of the coil, channel 123 is to the left of shape-forming wire 128, and in the sections disposed on the right-hand side of FIG. 6B and of the coil, channel 123 is to the right of the shape-forming wire.

In some embodiments, a distance of shape-forming wire 128 to longitudinal axis 107 of coil 106 remains fixed, throughout the coil, as illustrated by distances D_{wire} in FIG. 6B.

In some embodiments, and as illustrated in FIG. 6B, shape-forming wire 128 is disposed in the same position along the entire longitudinal length of elongate tube 122 (i.e., the tube does not rotate about its longitudinal axis), such that in a longitudinal cross-section of the coil, all the sections of shape-forming wire are disposed along two straight parallel lines, indicated by P in FIG. 6B.

In some embodiments, and as illustrated in FIG. 6B, shape-forming wire 128 is disposed in the same position along the entire longitudinal length of elongate tube 122 (i.e., the tube does not rotate about its longitudinal axis). When coil 106 has a fixed pitch, the vertical distance between a segment of shape-forming wire 128 in one loop of the coil and a segment in an adjacent loop of the coil is substantially fixed, as illustrated by distance Dₗₒₒₚ in FIG. 6B. Stated more broadly, and in a manner that is suitable also when the coil does not have a fixed pitch, the vertical distance between a segment of shape-forming wire 128 in one loop of the coil and a segment in an adjacent loop of the coil is equal to a sum of the pitch between the two adjacent loops and the exterior diameter of elongate tube 122.

FIG. 6B also illustrates the coil-radial direction, indicated by arrow 130, and the coil-axial direction, indicated by arrow 131. As explained in detail hereinabove, elongate tube 102, and coil 106 formed therefrom, have distinct mechanical properties in the coil-axial direction and in the coil-radial direction.

Reference is now made to FIGS. 7A and 7B, which are, respectively, a schematic perspective view illustration and a schematic sectional illustration of a coil 106′ formed from the elongate tube of FIGS. 3A and 3B, the coil being unsuitable for use in the medical system of FIGS. 1A and 1B.

As seen in FIGS. 7A and 7B, in coil 106′, the location of shape-forming wire 128 relative to elongate tube 122 changes along the coil. Stated differently, within coil 106′, elongate tube 122 rotates about its own longitudinal axis, and thus the relative position of channel 123 and of shape-forming wire 128 changes at different portions in the coil, and in different cross sections of the elongate tube within a cross section of the coil, as shown in FIG. 7B.

As a result, as seen in FIG. 7A, shape-forming wire 128 “travels” between the upper and lower portions of the loops of the coil.

In the illustration shown in FIG. 7B, in each loop of coil 106′, shape-forming wire 128 is disposed at approximately a 15 degree angle relative to its location in the previous loop of the coil, indicating that elongate tube 122 twists about its longitudinal axis.

In coil 106′, the distance between shape-forming wire 128 and longitudinal axis 107′ of the coil is different in different loops of the coil. Additionally, the shape-forming wire 128 is not disposed in the same position along the entire longitudinal length of elongate tube 122 (i.e., the tube rotates about its longitudinal axis), such that in a longitudinal cross-section of the coil, the sections of the shape-forming wire do not form two straight parallel lines.

It is to be appreciated that a coil, as shown in FIGS. 7A and 7B, is unlikely to be able to linearize properly for delivery through the working channel of a delivery tool (e.g., endoscope), as explained hereinabove, and that following linearization, kinks may form when the elongate tube re-coils. In some embodiments, the coil of FIGS. 7A and 7B may also not meet the flexure modulus requirements of coil 106, as described hereinabove with respect to FIGS. 1A and 1B, and as such would be unsuitable for use within system 100.

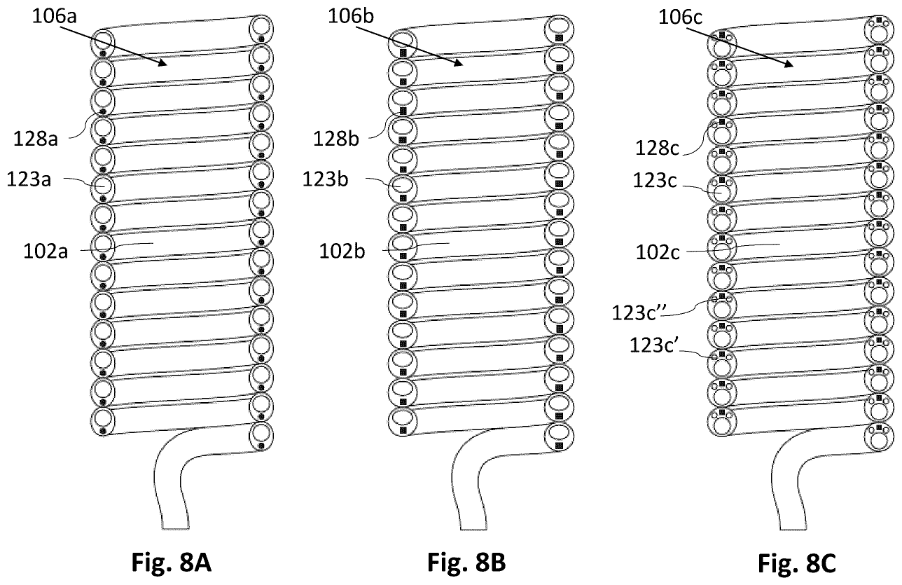

Reference is now made to FIGS. 8A, 8B, and 8C, which are schematic illustrations of exemplary structures of coils that can be formed of elongate tubes having a structure similar to that of elongate tube 122 of FIGS. 3A and 3B, which coils are suitable for use in the medical system of FIGS. 1A and 1B.

In an elongate tube 122a shown in FIG. 8A, the elongate tube has a substantially round cross-section, and includes shape-forming wire 128a having a generally square cross-section. As seen, shape-forming wire 128a is disposed at the lower end of the cross section of each loop of a coil 106a, such that elongate tube 122a does not rotate around itself throughout the length of the coil. Channel 123a of elongate tube 122a has a generally oval cross-section, the cross-section having a long axis which is substantially perpendicular to the coil-axis, and is disposed above the shape-forming wire in each loop of the coil.

In an elongate tube 122b shown in FIG. 8B, the elongate tube has a substantially oval cross-section, and includes shape-forming wire 128b having a generally circular cross-section. As seen, shape-forming wire 128b is disposed at the lower end of the cross section of each loop of a coil 106b, such that elongate tube 122b does not rotate around itself throughout the length of the coil. Channel 123b of elongate tube 122b has a generally circular cross-section, and is disposed above the shape-forming wire in each loop of the coil.

In an elongate tube 122c shown in FIG. 8C, the elongate tube has a substantially circular cross-section, and includes shape-forming wire 128c having a generally square cross-section. As seen, shape-forming wire 128c is disposed at the upper end of the cross section of each loop of a coil 106c, such that elongate tube 122c does not rotate around itself throughout the length of the coil. Elongate tube 122c includes three channels, labeled 123c, 123c′, and 123c″, each having a substantially circular cross-section, with channel 123c having a greater diameter than channels 123c′, and 123c″. As seen, in the cross-section of each loop of the coil, channel 123c is disposed directly beneath shape-forming wire 128c, and channels 123c′ and 123c″ are disposed on either side of the shape-forming wire, slightly toward channel 123c.

It is to be appreciated that each of elongate tube 122a, 122b, and 122c shown in FIGS. 8A to 8C would be able to meet the flexure modulus requirements described hereinabove with respect to FIGS. 1A and 1B, and as such would be able to form coils within the lumen of the GI tract, and the formed coils would be suitable for use within system 100.

Reference is now made to FIG. 9, which is a perspective sectional illustration of an exemplary structure of an elongate tube 132, similar to elongate tube 102 of FIGS. 1A and 1B, according to embodiments of the disclosed technology. Elongate tube 132 has different flexure modulus values along different axes thereof, as described hereinabove with respect to elongate tube 102 of FIGS. 1A and 1B.

Elongate tube 132 includes a plurality of orifices including a first subset of orifices 134a, and a second subset of orifices 134b. Elongate tube 132 defines a first, vacuum-delivery, channel 133 in fluid communication with a first subset of orifices 134a, and a second channel 136 in fluid communication with a second subset of orifices 134b. Second channel 136 may function as a fluid-delivery channel for delivering a fluid, such as a periodically delivered flushing fluid or a constant irrigation fluid to the vicinity of elongate tube 132, via orifices 134b. However, in some other embodiments, second channel 136 may function as a second vacuum-delivery channel.

In use of system 100, first channel 133 and the first subset of orifices 134a are in fluid communication with the source of negative pressure 110, such that fluid from the vicinity of the orifices in the first subset is drained, via those orifices and first channel 133. Second channel 136 and orifices 134b in the second subset are in fluid communication with source 116 of fluid 118 (FIG. 1A), such that fluid 118 supplied from source 116 flows through second channel 136 and orifices 134b into the vicinity of the orifices, such as into esophagus 10 in the vicinity of wound 12. The fluid may be supplied continuous, intermittently, periodically or as needed.

A filament or wire-accommodating channel 137 is also formed in the wall of elongate tube 132, and is adapted to accommodate a shape-forming wire or monofilament 138, adapted to form the coil in the resting operative state of elongate tube 132. Shape-forming wire 138 may be similar to shape-forming wire 128, described hereinabove with respect to FIGS. 3A and 3B, and has similar mechanical and structural properties thereto.

Reference is now made to FIG. 10, which is a planar sectional illustration of an exemplary structure of an elongate tube 142, similar to elongate tube 102 of FIGS. 1A and 1B, according to embodiments of the disclosed technology. Elongate tube 142 has different flexure modulus values along different axes thereof, as described hereinabove with respect to elongate tube 102 of FIGS. 1A and 1B.

Elongate tube 142 includes a central channel 143, which is in fluid communication with orifices similar to orifices 104 of FIGS. 1A and 1B (not explicitly shown). An exterior surface of elongate tube 142 includes a plurality of troughs 145, extending longitudinally along the tube. In some embodiments, each of the orifices of elongate tube 142 spans the width of multiple troughs 145. Stated differently, the width of troughs 145 is smaller than the diameter of the orifices. In other embodiments, each of the orifices of elongate tube 142 is in fluid communication with a single trough 145, for example by each orifice having a diameter smaller than a width of the trough.

Channel 143 is in fluid communication with source 110 of negative pressure, and functions substantially as described hereinabove with respect to channel 123 of FIGS. 3A and 3B. The negative pressure is applied to the vicinity of the tube 142 via the orifices, and drains fluid and debris from the vicinity of the tube.

Troughs 145 fulfill multiple purposes in the treatment using the system of the disclosed technology. The presence of troughs 145 assists in maintaining the orifices open, particularly when loops 106 (FIGS. 1A and 1B) of the elongate tube are disposed directly one over the other, with no gaps. In such conditions, troughs 145 form a channel through which the negative pressure can be applied to the vicinity, even if the loops engage one another. Additionally, troughs 145, which have a narrow cross section, are delineated by ridges, which ridges can engage the surrounding tissue, such as tissue of esophagus 12, and promote tissue growth, thereby to accelerate healing. Furthermore, in some embodiments, fluid may be drained via troughs 145 into the orifices, thus facilitating drainage from a larger area using fewer orifices, and the orifices are less likely to be blocked or occluded by debris.

In some embodiments, elongate tube 142 may optionally further include a fluid-delivery channel 146 associated with fluid-delivery orifices (not explicitly shown) similar to orifices 134b of FIG. 9, for delivery of fluid to the vicinity of the elongate tube 142. In some embodiments, and as illustrated, fluid-delivery channel 146 may be formed in the circumferential wall of elongate tube 142.

In some embodiments, elongate tube 142 may further include a wire-accommodating channel 147, formed in the circumferential wall of elongate tube 142, substantially as described hereinabove with respect to FIGS. 3A and 3B. Wire-accommodating channel 147 is adapted to accommodate a shape-forming wire or microfilament, substantially as described hereinabove with respect to shape-forming wire 128. In some embodiments, in addition to or instead of a shape-forming wire, elongate tube 142 may further include a monofilament, similar to monofilament 111 described hereinabove with respect to FIG. 2. The presence of such a monofilament will assist in providing the mechanical properties required for the elongate tube, with respect to flexure moduli in the axial and radial directions.

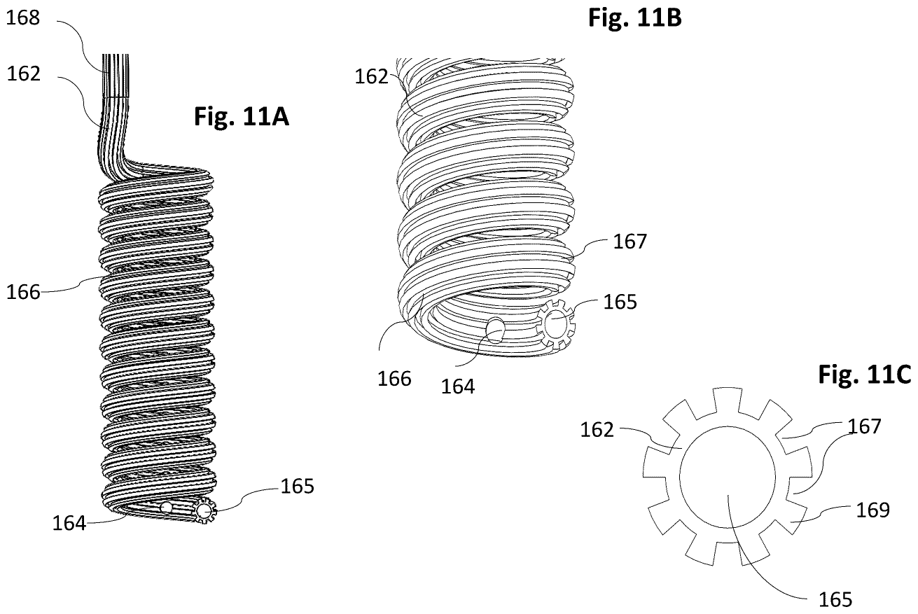

Reference is now additionally made to FIGS. 11A, 11B, and 11C, which are, respectively, side view illustrations and an end view illustration of an exemplary structure of an elongate tube 162, similar to elongate tube 102 and suitable for forming part of the medical system of FIGS. 1A and 1B, according to embodiments of the disclosed technology. Elongate tube 162 has different flexure modulus values along different axes thereof, as described hereinabove with respect to elongate tube 102 of FIGS. 1A and 1B.

Elongate tube 162, which has a similar function to elongate tube 102 of FIGS. 1A and 1B, but differs therefrom in several aspects. Like elongate tube 102, which includes orifices 104 and forms a coil including a plurality of loops 106, elongate tube 162 includes orifices 164 and forms a coil including a plurality of loops 166.

Elongate tube 162 includes an internal channel 165, and in addition includes one or more exterior channels 167, here shown as a plurality of troughs extending longitudinally along an exterior surface of the elongate tube. Orifices 164, which, in the embodiment of FIGS. 11A to 11C, are disposed about a single circumference of the elongate tube, adjacent the distal end of the elongate tube, are in fluid communication with interior channel 165 as well as with at least one of exterior channels 167, and facilitate fluid flow between the internal channel and at least one of the exterior channels. However, in some embodiments, orifices 164 may be longitudinally distributed along a portion of the elongate tube or along the entirety of the elongate tube, for example as shown in FIGS. 1A and 1B. In the illustrated embodiment, fluid-tight lumen 168 has a similar structure to elongate tube 162, and also includes exterior channels.

As seen in FIG. 11B, in some embodiments, each of orifices 164 spans the width of multiple troughs, or exterior channels, 167. Stated differently, the cross section or diameter of troughs 167 is smaller than the diameter of orifices 164.

Channel 165 is in fluid communication with source 110 of negative pressure, via fluid tight lumen 168, and functions substantially as described hereinabove with respect to FIGS. 1A and 1B. The negative pressure is applied to the vicinity of the elongate tube 162 via orifices 164 and troughs 167, and drains fluid and debris from the vicinity of the tube, via troughs 167 and orifices 164, into channel 165.

It is to be appreciated that in some embodiments, orifices 164 may be disposed about a single circumference of elongate tube 162, adjacent the proximal end of the elongate tube. In some such embodiments, internal channel 165 must extend along a proximal longitudinal portion of the elongate tube leading up to, or slightly past, orifices 164, but need not necessarily extend beyond orifices 164. In such embodiments, negative pressure would be delivered from fluid-tight lumen, via the portion of internal channel 165 and the orifices 164 to a proximal end of troughs 167, such that fluid and debris from the vicinity of the coil is drawn longitudinally along troughs 167 from the distal end toward the proximal end, and from there through orifices 164 into internal channel 165.

Troughs 167 fulfill multiple purposes in the treatment using the system of the disclosed technology. The presence of troughs 167 assists in maintaining orifices 164 open, particularly when loops 166 of the elongate tube are disposed directly one over the other, with no gaps. In such conditions, troughs 167 may form a channel through which the negative pressure can be applied to the vicinity, even if the coils engage one another. Additionally, troughs 167, which have a narrow cross section, are delineated by ridges 169. These ridges provide a texture to the exterior surface of elongate tube 162, and can engage the surrounding tissue, such as tissue of esophagus 12, to promote tissue growth, thereby to accelerate healing. Furthermore, in some embodiments, fluid may be drained via troughs 167 into orifices 164, thus facilitating drainage from a larger area using fewer orifices, and the orifices are less likely to be blocked or occluded by debris.

In some embodiments, elongate tube 162 may optionally further include a second channel associated with fluid delivery orifices (not explicitly shown) for delivery of fluid to the vicinity of the elongate tube 162, substantially as described hereinabove with respect to FIGS. 1A and 1B.

It is to be appreciated that elongate tube 162 may additionally include a wire-accommodating channel, similar to channel 127 of FIGS. 3A and 3B and/or a shape-forming wire or monofilament, similar to wire shape-forming 128 of FIGS. 3A and 3B. The structure and functionality of such a wire-accommodating channel and/or such a shape-forming wire would be similar to that described hereinabove with respect to channel 127 and shape-forming wire 128.

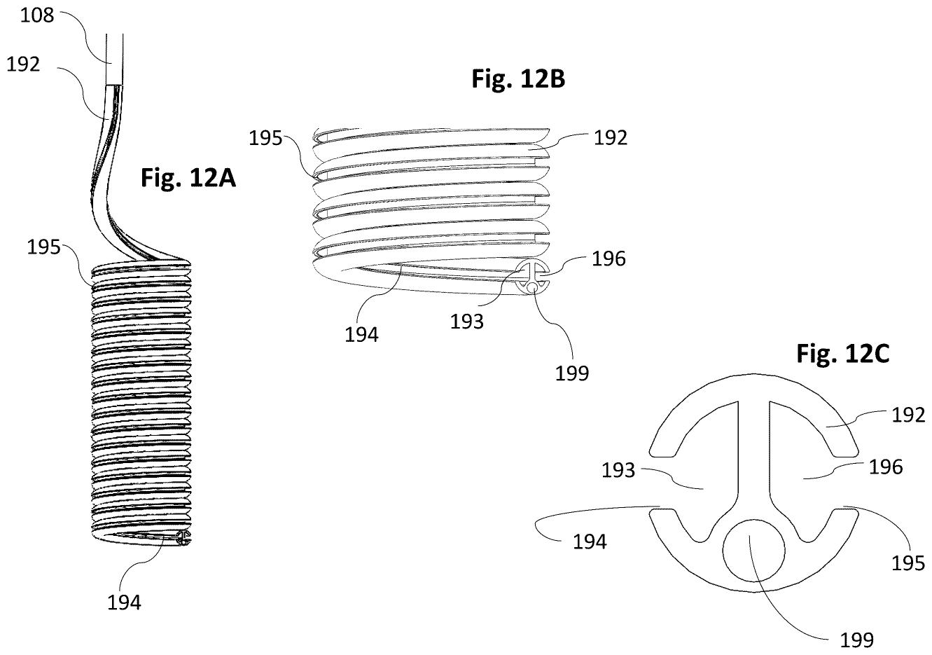

FIGS. 12A, 12B, and 12C, are schematic illustrations of exemplary structures of a coil formed of an elongate tube 192, similar to elongate tube 132 of FIG. 9 and suitable for forming part of the medical system of FIGS. 1A and 1B, according to embodiments of the disclosed technology. Elongate tube 192 has different flexure modulus values along different axes thereof, as described hereinabove with respect to elongate tube 102 of FIGS. 1A and 1B. However, elongate tube 192 is different from elongate tubes 102 and 132 in several aspects.

Like elongate tube 132, which includes channels 133 and 136 and forms a coil including a plurality of loops 106, elongate tube 192 includes one or more channels, here shown as a pair of channels 193 and 196 and forms a coil including a plurality of loops. However, in elongate tube 192, the orifices are replaced with longitudinal slots 194 and 195, which function as the portals facilitating fluid communication between channels 193 and 196 and the environment outside of elongate tube 192.

In some embodiments, each of channels 193 and 196 is in fluid communication with source 110 of negative pressure, via fluid tight lumen 108, and functions substantially as described hereinabove with respect to channel 133 of FIG. 9 and as described hereinabove with respect to elongate tube 102 of FIGS. 1A and 1B. The negative pressure is applied to the vicinity of the elongate tube 192 via slots 194 and 195, and drains fluid and debris from the vicinity of the tube, via the slots, into channels 193 and 196.

In some embodiments, channel 193 is in fluid communication with source 110 of negative pressure, and functions substantially as described hereinabove with respect to channel 133 of FIG. 9 and as described hereinabove with respect to elongate tube 102 of FIGS. 1A and 1B. At the same time, channel 196 is in fluid communication with source 116 of fluid, and functions as a fluid-delivery channel, in a similar manner to channel 136 of FIG. 9.

In some embodiments, elongate tube 192 may further include a third channel 199. In some embodiments, channel 199 may be associated with fluid delivery orifices (not explicitly shown) for delivery of fluid to the vicinity of the elongate tube 192, substantially as described hereinabove with respect to channel 136.

In some other embodiments, channel 199 may function as a wire-accommodating channel, in a similar manner to channel 127 described hereinabove with respect to FIGS. 3A and 3B. In such embodiments, a shape-forming wire or monofilament (not explicitly shown) similar to shape-forming wire 128 may extend through channel 199.

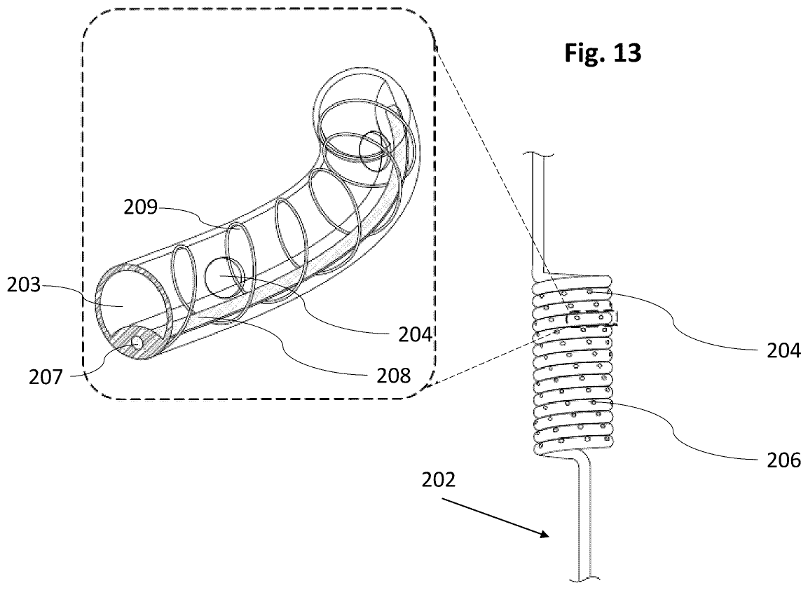

Reference is now made to FIG. 13, which is a schematic illustration of an exemplary structure of a reinforced elongate tube 202, similar to elongate tube 122 and suitable for forming part of the medical system of FIGS. 1A and 1B, according to embodiments of the disclosed technology. Elongate tube 202 has different flexure modulus values along different axes thereof, as described hereinabove with respect to elongate tube 102 of FIGS. 1A and 1B.

As seen, elongate tube 202 is substantially similar to elongate tube 122 of FIGS. 3A and 3B, and includes a channel 123 and a plurality of orifices 124. Elongate tube 202 is configured to form a coil including loops 206, as shown. Additionally, elongate tube 202 includes a wire-accommodating channel 207, similar to channel 127 of FIGS. 3A and 3B, which accommodates a shape-forming wire 208 or monofilament therein, as described hereinabove.

It is a particular feature of elongate tube 202 that a reinforcing wire 209 extends through the material of the elongate tube, typically in a helical manner, in addition to shape-forming wire 208. Reinforcing wire 209 is adapted to assist in ensuring that elongate tube 202 is sufficiently resistant to application of negative pressure, and to external pressures that may occur within the gastrointestinal tract, such as during peristalsis, so that the elongate tube retains its coiled shape under such pressured conditions. In some embodiments, a direction of rotation of helical reinforcing wire 209 is opposite to the direction of rotation of loops 206 of the coil formed by the elongate tube. As such, if the coil extends in a clockwise direction, the helix of reinforcing wire 209 would extend in a counterclockwise direction.

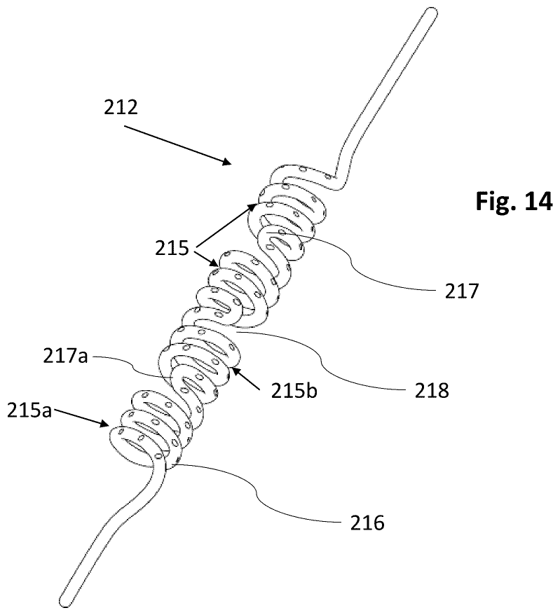

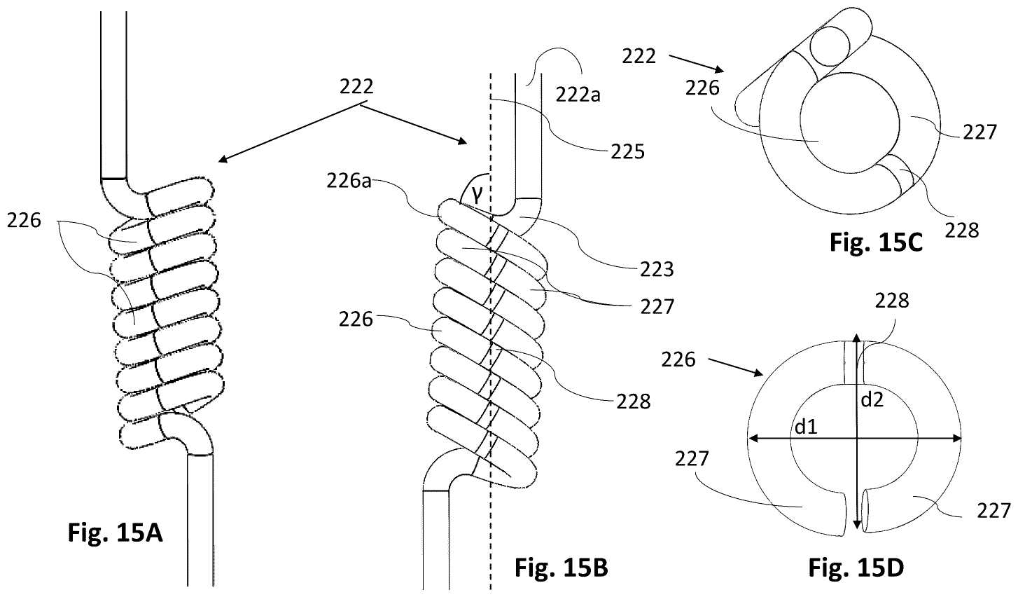

Reference is now made to FIG. 14, which is a schematic illustration of an exemplary coil structure of an elongate tube 212 suitable for forming part of the medical system of FIGS. 1A and 1B, according to embodiments of the disclosed technology. Elongate tube 212 may have an internal structure or a cross-sectional structure similar to any one of elongate tubes 102, 122, 122a, 122b, 122c, 132, 142, 162, 192, or 202 described hereinabove.

As seen in FIG. 14, elongate tube 212 forms a three-dimensional structure including multiple coil sections 215, where each coil section includes multiple loops 216. Each pair of adjacent coil sections 215 are oriented in opposite directions, such that a hair-pin-like bend 217 is formed between each two adjacent sections. For example, when looking from the distal end 212b of elongate tube 212 toward the proximal end 212a thereof, the direction of rotation in coil section 215a is clockwise, and the direction rotation in coil section 215b is counterclockwise, with the two sections being connected by hair-pin-like bend 217a.

In some embodiments, such as that shown in FIG. 14, elongate tube 212 includes multiple sections 215 of clockwise oriented loops and counterclockwise oriented loops, such that the total number of clockwise oriented loops is equal to the total number of counterclockwise oriented loops. In some such embodiments, each section may have a different number of loops.

In some embodiments, the three-dimensional structure of elongate tube 212 may be advantageous over a simple coiled structure, as shown for example in FIGS. 1A and 1B, since the structure of tube 212 is more resistant to deformation when external forces are applied thereto, such as during peristaltic action in the gastrointestinal tract. For example, when the coil of FIGS. 1A and 1B is held at opposing ends thereof and is pulled along the longitudinal coil axis 107, the elongate tube tends to twist about its own axis. The introduction of clockwise and counterclockwise oriented loops prevents such twisting. As a result of twisting being prevented, the stability of the coil is maintained when axial forces are applied thereto, since the coil can more readily return to its coiled state without introduction of unwanted twists or kinks.

Additionally, bends 217 result in the formation of gaps 218 in remaining portions of the circumference of the coil, across from the bends. When negative pressure is applied to the vicinity of elongate tube 212, as described hereinabove, tissue of the lumen in which the elongate tube is disposed can be drawn into gaps 218, resulting in the formation of multiple separate vacuum chambers in a similar manner to that described hereinabove with respect to FIG. 1B.

Reference is now made to FIGS. 15A, 15B, 15C, and 15D, which are, respectively, right and left side view illustrations, a top view illustration, and a segmented illustration an exemplary structure of an elongate tube 222, suitable for forming part of the medical system of FIGS. 1A and 1B, according to embodiments of the disclosed technology. Elongate tube 222 may have an internal structure or a cross-sectional structure similar to any one of elongate tubes 102, 122, 122a, 122b, 122c, 132, 142, 162, 192, or 202 described hereinabove.

As seen in FIGS. 15A and 15B, elongate tube 222 forms a three-dimensional structure including a coil including multiple loops 226. As seen clearly in FIG. 15D, which illustrates a planar view of single loop of the coil elongate tube 222, each loop 226 is pre-formed to include a pair of substantially hemispherical portions 227 separated by a substantially linear portion 228. As a result, when the loop is viewed in a single plane, as shown in FIG. 15D, the loop has two orthogonal dimensions d1 and d2, where one dimension is greater than the other, such that hollow in the center of the loop is substantially oval or elliptical. In the illustrated embodiment, d1>d2.

When in the coil form, stability of elongate tube 222 requires that the tube have a substantially circular outer perimeter, as seen in FIG. 15C. As a result of the presence of linear portions 228 in loops 226, in the coiled form as shown in FIGS. 15A and 15B, each of loops 226 is tilted relative to longitudinal coil axis 225 extending through the center of the coil. In some embodiments, an angle γ between a loop 226 and coil axis 225 is in the range of 45-85 degrees, in the range of 50-75 degrees, or in the range of 55-65 degrees. Additionally, shoulder 223 formed between the first loop 226a of the coil and a proximal end 222a of elongate tube 222 may be in the range of 0-60 degrees offset from the longitudinal axis of the coil.

It is to be appreciated that elongate tube 222 is pre-formed to generate a coil including loops 226 as illustrated, for example by suitable treatment of a shape-forming wire disposed within the elongate tube. As such, elongate tube 222 forms a coil in which loops 226 are tilted relative to longitudinal coil axis 225 when the coil is at rest, i.e., without any extraneous force being applied directly thereto (e.g., gravitational forces or forces applied by presence of sub-atmospheric pressure) other than the forces naturally applied by gravity and by the presence of atmospheric pressure.

In some embodiments, the structure of elongate tube 222, in the coil form, may reduce the tolerance of the coil to externally applied radial forces, such as those applied by the tissue onto the coil when negative pressure is applied through the coil. While the tolerance remains sufficient to maintain the coil shape, the reduced tolerance to radial forces ensures that the coil may not stack as tightly, or may slightly collapse, within the lumen of the gastrointestinal tract during application of negative pressure to the lumen. Such change to the structure of the coil within the GI tract can assist in preventing the negative pressure, applied via the portals to the tissue of the lumen, from being applied constantly to a specific point of the tissue. As such, the risk of damage to the tissue caused by extended application of negative pressure to a specific area of the tissue is reduced.

The following description relates to elongate tubes 102, 122, 122a, 122b, 122c, 132, 142, 162, 192, 202, 212, and 222 shown in FIGS. 1A-15D, and to fluid-tight lumen 108 shown in FIGS. 1A to 2. While the description uses reference numerals provided with respect to FIGS. 1A and 1B, it is to be appreciated that it is similarly applicable to the same elements of FIGS. 3A to 15D, even if those elements are designated by different reference numerals. Similarly, the following description relates to orifices 104 as the portals facilitating fluid communication between the elongate tube and the surrounding environment. However, the description provided for orifices 104 is similarly applicable to other types of portals, such as slots 194 and 195.

In some embodiments, elongate tube 102 has a diameter d, shown in FIG. 1A, in the range of 1 mm to 8 mm, 1 mm to 5 mm, or 2 mm to 4 mm.

In some embodiments, elongate tube 102 may be formed of a porous material, such as ePTFE, PTFE-foam, EVA, PU-foam, and PP-foam. In some embodiments, orifices 104, may be or may include pores in the porous material.

In some embodiments, elongate tube 102 may comprise, or may consist of, a radiopaque marker, radioactive marker, magnetic marker, and/or magnetic resonance marker. In some embodiments, elongate tube 102 may comprise, or may consist of, a metal, a natural or elastic polymer, a plastic, a shape memory alloy, a super elastic alloy, and/or a biocompatible material, a biodegradable material, a bioresorbable material, and/or a bioabsorbable material.

In some embodiments, elongate tube 102 may be formed of a material relatively unlikely to irritate the gastrointestinal tract.

In some embodiments, elongate tube 102 may configured for an antimicrobial or anti-inflammatory effect. In some embodiments, elongate tube 102 comprises an antimicrobial or anti-inflammatory material. In some embodiments, elongate tube 102 is pretreated or coated with an antimicrobial or anti-inflammatory agent.

In some embodiments, elongate tube 102 is adapted to deliver to the vicinity of wound 12 an antimicrobial or anti-inflammatory medicament for treatment of the wound. For example, this may be accomplished by delivery of a medicament fluid via the channel and orifices of the elongate tube, as described hereinabove.

In some embodiments, elongate tube 102 has a textured exterior surface adapted to frictionally engage an interior surface of the gastrointestinal tract. An example of such a textured exterior surface is illustrated in FIGS. 4 to 5C, and its advantages are described hereinabove.

In some embodiments, elongate tube 102 may comprise, or may consist of, a shape memory material, an elastic material, a super-elastic material, or another polymeric material, adapted to direct or result in formation of a predetermined coil shape. In some embodiments, the predetermined coil shape has a longitudinal resilience, despite having a degree of collapsibility and expandability, in the first, resting operative state. It is understood that various mechanical properties of the material(s) used to form elongate tube 102, including brittleness, ductility, elasticity, hardness, malleability, plasticity, strength, and toughness, may be suitably selected to direct formation of the coil shape.

In some embodiments, the elongate tube 102 may have any suitable cross-sectional shape, including a circular cross section, an oval cross section, a D-shaped cross section, an I-shaped cross section, or a rectangular cross section.

In some embodiments, elongate tube 102 may have embedded therein, or extending through a dedicated channel in the tube, one or more shape-forming wires, such as shape-forming wires 128 and 138 shown and described with respect to FIGS. 3A to 9.

In some embodiments, elongate tube 102 may have embedded therein one or more reinforcing wires, such as wire 209 shown and described with respect to FIG. 13.

The channel(s) within elongate tube 102 have any suitable shape or cross section. For example, the channel(s) may have a circular cross section or a polygonal cross section in a direction perpendicular to a longitudinal axis of the elongate tube. In embodiments in which elongate tube 102 includes more than one channel, the channels need not have the same cross section, in a direction perpendicular to the longitudinal axis of the elongate tube, as seen clearly in FIGS. 3A and 3B.

In some embodiments, the channel(s) may extend along the entire longitudinal length of elongate tube 102. In other embodiments, the channel(s) may extend only within or along a proximal portion of the elongate tube 102, coupled to fluid-tight lumen 108, but may not span the entire longitudinal length of the elongate tube.

In some embodiments, the elongate tube includes multiple channels, a first (e.g., 133 in FIG. 9) connected to source of negative pressure 110 and another (e.g., 136 in FIG. 9) connected to source of fluid 116, such that negative pressure and fluid may be provided to the vicinity of elongate tube 102, via different channels or orifices, substantially simultaneously, or at different times via different channels.

In some embodiments, the cross-sectional shape of the elongate tube, of the shape-forming wire, and/or of the channel(s) may be selected to improve mechanical characteristics of the elongate tube to the functionality for which it is used. In some embodiments, the cross-sectional shape of the elongate tube, of the shape-forming wire, and/or of the channel(s) may be selected to reduce deformation of the elongate tube, and to increase repeatability on cycle hysteresis. In some embodiments, the cross-sectional shape of the elongate tube, of the shape-forming wire, and/or of the channel(s) may be selected to reduce plastic deformation at high strains. In some embodiments, the cross-sectional shape of the elongate tube, of the shape-forming wire, and/or of the channel(s) may be selected to facilitate thermoforming of the tube, as described herein. In some embodiments, the cross-sectional shape of the elongate tube, of the shape-forming wire, and/or of the channel(s) may be selected to reduce elongation of the elongate tube during pushing and/or pulling thereof.

In some embodiments, the composition and shape of the shape-forming wire, or coil frame, is adapted to provide reinforcement to maintain the size and shape of the coil. The composition and shape of the coil frame may also provide flexibility to the loops, and to the coil as a whole, to permit stretching and compressing of the coil while preventing formation of kinks. In some embodiments, the shape-forming wire comprises a material having a thermal-shaping temperature which does not substantially affect the elongate body structure. In some embodiments, the thermo-shaping temperature is lower than a melting point of a material from which the elongate tube body is formed.

In some embodiments, the shape-forming wire has an elastic range greater than 0.5%.

In some embodiments, orifices 104, are disposed about a single circumference of the elongate tube, for example as shown in FIGS. 11A and 11B. In some embodiments, the portals can be disposed along a single longitudinal position along the longitudinal axis of elongate tube 102 (as shown in FIG. 1A), such that when the elongate tube is uncoiled, the orifices form a straight line along the length of the tube. In some embodiments, the portals may be disposed along multiple longitudinal positions along the longitudinal axis of the elongate tube.

In some embodiments, at least one longitudinal channel extends along an external longitudinal portion of the elongate tube, as shown in FIGS. 11A and 11B.

In other embodiments, orifices 104 are disposed longitudinally, along a longitudinal length of elongate tube 102, or at different longitudinal positions along the longitudinal axis. In other embodiments, the orifices may be distributed about a single circumference of elongate tube 102.

In some embodiments, and as shown in FIG. 1A, orifices 104 may be equidistantly distributed along or about elongate tube 102. In other embodiments, orifices 104 may be heterogeneously distributed along or about elongate tube 102. For example, a first pair of adjacent orifices 104 may have a first longitudinal distance therebetween, and a second pair of adjacent orifices 104 may have a second longitudinal distance therebetween, the second longitudinal distance being different from the first longitudinal distance. As another example, a first pair of adjacent orifices 104 may have a first circumferential distance therebetween, and a second pair of adjacent orifices 104 may have a second circumferential distance therebetween, the second circumferential distance being different from the first circumferential distance.

In some embodiments, each of orifices 104 has substantially the same diameter. In some other embodiments, orifices 104 in a first subset of the orifices have a first diameter, and orifices 104 in a second subset of the orifices have a second diameter, the second diameter being different from the first diameter.

In the embodiment, the cross-sectional area of the orifices 104 increases along the length of the elongate tube, or of the coil, from the proximal end towards the distal end. In some such embodiments, the cross-sectional area of the distal-most orifice is at least 50% greater than the cross-sectional area of the proximal-most orifice.

In some embodiments, a greatest dimension of each of orifices 104 is within the range of 0.5 mm to 10 mm, 0.5 mm to 8 mm, 0.5 mm to 5 mm, 0.5 mm to 3 mm, or 1 mm to 2 mm.

In some embodiments, in the resting operative state of elongate tube 102, at least some of orifices 104 are oriented inwardly, toward a center of the coil, for example as shown in FIG. 10A. In some embodiments, in the resting state of elongate tube 102, at least some of orifices 104 are oriented outwardly, away from the center of the coil, for example as shown in FIGS. 1A and 1B.

In some embodiments, in the resting operative state, elongate tube 102 is substantially devoid of orifices 104 oriented outwardly, away from a center of the coil, such that all of orifices 104 are oriented toward the center of the coil. In some embodiments, in the resting operative state, elongate tube 102 is substantially devoid of orifices 104 oriented inwardly, toward a center of the coil, such that all of orifices 104 are oriented outwardly and away from the center of the coil.

In some embodiments, in the first, resting operative state of elongate tube 102, an axial length of the coil, indicated in FIG. 1A by L, is at least 10 mm, at least 15 mm, at least 20 mm, at least 25 mm, at least 30 mm at least 35 mm, at least 40 mm, at least 50 mm, at least 60 mm, or at least 80 mm.

In the context of the present application and claims, the axial length of the coil is defined as the length of the coil as measured along a longitudinal axis, extending through the center of the coil. The axial length of the coil is, by definition, shorter than the longitudinal length of elongate tube 102, of which the coil is formed. A ratio between the axial length of the coil and the longitudinal length of the elongate tube depends on the diameter of each loop of the coil, and on the tightness, or pitch, of the coil.

In some embodiments, in the first, resting operative state of elongate tube 102, axial length L of the coil is at most 200 mm, at most 150 mm, at most 100 mm, at most 80 mm, at most 70 mm, at most 60 mm, or at most 50 mm.

In some embodiments, in the first, resting operative state of elongate tube 102, axial length L of the coil is in the range of 10 mm to 200 mm, 10 mm to 150 mm, 10 mm to 120 mm, 10 mm to 100 mm, 10 mm to 80 mm, 10 mm to 70 mm, 10 mm to 60 mm, 10 mm to 50 mm, 20 mm to 50 mm, 30 mm to 50 mm, 40 mm to 50 mm, 10 mm to 40 mm, 15 mm to 40 mm, 10 mm to 35 mm, or 15 mm to 35 mm.

In some embodiments, elongate tube 102 has a second, draining operative state, when negative pressure, is applied to the elongate tube. In the second draining operative state, the coil has a second axial length L2, which is not greater than axial length L. In some embodiments, in the draining operative state, the second axial length L2 in the range of 10 mm to 50 mm, 10 mm to 40 mm, 20 mm to 50 mm, or 20 mm to 40 mm.

In some embodiments, fluid-tight lumen 108 and/or elongate tube 102, and the coil formed thereby, are adapted to have a negative pressure in the range of 25-350 mmHg, 30-350 mmHg, 40-350 mmHg, 50-350 mmHg, 60-350 mmHg, 70-350 mmHg, 80-350 mmHg, to 100-350 mmHg applied thereto.

In some embodiments, during application of negative pressure to elongate tube 102 and to the coil formed thereby, loops 106 of the coil tilt relative to the longitudinal axis of the coil (indicated by reference numeral 107 in FIG. 1A). Such tilting of the loops 106 causes the cross-sectional area of each ring to change, for example from having a circular cross-sectional area to having an oval cross-sectional area. In some embodiments, each of loops 106 tilts, to the right or to the left, by up to 50 degrees, up to 45 degrees, up to 30 degrees, or up to 20 degrees from longitudinal axis 107. Typically, all the loops tilt in the same direction, and remain substantially parallel to each other. It is a particular feature of the disclosed technology that even when the loops 106 tilt relative to longitudinal axis 107, the coil remains distributed about the same longitudinal axis.

In some embodiments, a L:L2 ratio between axial length L of the coil in the resting operative state, and second axial length L2 of the coil in the draining operative state is in the range of 1:1 to 4:1, 1:1 to 3:1, 1:1 to 2:1, 1:1 to 1.5:1, or 1:1 to 1.25:1 as measured in an ex-vivo female pig esophagus, of a pig weighing approximately 60 kg or in an in vitro model thereof.

In some embodiments, a difference between the cross-sectional diameter D of the coil, or in the cross sectional area of the coil, in the resting operative state and the second, draining, operative state, when negative pressure, for example in the range of 25-350 mmHg or 50-350 mmHg, is applied to elongate tube 102, is less than 75%, less than 50%, less than 20%, or less than 10%, of the cross-sectional diameter D or the cross sectional area in the resting operative state, as measured in an ex-vivo pig esophagus, of a pig weighing 60 kg or in an in vitro model thereof.

In some embodiments, a difference between axial length L of the coil in the resting operative state, and axial length L2 of the coil in the draining operative state is not greater than 150 mm, not greater than 125 mm, not greater than 125 mm, not greater than 100 mm, not greater than 75 mm, not greater than 50 mm, not greater than 40 mm, not greater than 30 mm, not greater than 20 mm, or not greater than 10 mm.

In some embodiments, each of the loops of the coil is on a separate plane, with the planes optionally being parallel to each other and/or having a gap, or pitch, therebetween.

In some embodiments, in the first, resting, operative state of elongate tube 102, the coil has a uniform pitch between each pair of adjacent loops.

In some embodiments, in the first operative state, a pitch of the coil is in a range of 2.5 mm to 25 mm. In some embodiments, in the first operative state, a pitch of the coil is in a range of 5 mm to 25 mm. In some embodiments, in the first operative state, a pitch of the coil is in a range of 2 mm to 40 mm. In some embodiments, in the first operative state, a pitch of the coil is in a range of 2 mm to 30 mm. In some embodiments, in the first operative state, a pitch of the coil is in a range of 2 mm to 25 mm. In some embodiments, in the first operative state, a pitch of the coil is in a range of 2 mm to 20 mm. In some embodiments, in the first operative state, a pitch of the coil is in a range of 3 mm to 15 mm.

In some embodiments, in the first, resting, operative state of elongate tube 102, the coil has a first pitch P. In some embodiments, in the second, draining, operative state of elongate tube 102, the coil has a second pitch P2. In some embodiments, a ratio between first pitch P and second pitch P2 is in the range of 1:1 to 6:1.

In some embodiments, the pitches P and/or P2 of the coil facilitate contraction and expansion of the coil in response to contraction and expansion of the gastrointestinal tract in which the coil is positioned, such that a three-dimensional position of the coil within the gastrointestinal tract, is maintained during motion of the gastrointestinal tract. For example, the position of the coil may be maintained to motion within the gastrointestinal tract, even during peristaltic contractions of the f.

In some embodiments, at least one characteristic of the coil is configurable by making a change to a condition in an environment surrounding the coil. In some embodiments, the characteristic may be, or may include, a chemical characteristic or a mechanical characteristic of the coil.

For example, the changed condition may be a temperature of the coil, or a temperature in the vicinity of the coil during the deployment.

In some embodiments, in the first, resting state of elongate tube 102, a number of loops 106 in the coil formed from the elongate tube is at least 3, at least 4, at least 5, at least 8, or at least 10.

In some embodiments, in the first, resting operative state of elongate tube 102, the number of loops 106 in the coil is within the range of 3 to 15, 5 to 15, 5 to 12, 8 to 12, 3 to 30, 5 to 30, 5 to 30, or 8 to 30.

In some embodiments, in the first, resting operative state of elongate tube 102, the diameter of each loop 106 in the coil or the diameter of the coil as a whole, indicated by D in FIG. 1A, is in a range of 0.5 cm to 5 cm, 1 cm to 5 cm, 0.5 cm to 4 cm, 1 cm to 4 cm, 2 cm to 4 cm, 0.5 cm to 3 cm, 1 cm to 3 cm, 1 cm to 2.5 cm, 1 cm to 2 cm or 2 cm to 3.5 cm.

In some embodiments, in the first, resting operative state of elongate tube 102, the diameter of at least one of loops 106, and in some embodiments of each loop 106, in the coil is not greater than 1.5 cm.

In some embodiments, a ratio of diameters between the cross-sectional diameter D of the coil in the operative state and the diameter of the elongate tube in the delivery state is 1:3 to 1:15, 1:3 to 1:10, 1:3 to 1:7, or 1:4 to 1:6.

In some embodiments, the coil has an inner diameter Di surrounding the inner volume or cavity of the coil through which additional tube 119 can pass (see FIG. 1). In some embodiments, inner diameter Di is not greater than 26 mm, not greater than 23 mm, not greater than 20 mm, not greater than 15 mm, or not greater than 10 mm.

In some embodiments, and as shown in FIG. 1A, all of loops 106 have substantially the same external diameter.

In some embodiments, at least two loops 106 have substantially the same diameter. In some embodiments, the two loops having substantially the same diameter are a proximal-most loop, and a distal-most loop of the coil. In some embodiments, the diameter of loops 106 other than the proximal-most loop and the distal-most loop is not greater than the diameter of the proximal-most loop. In some embodiments, the diameter of loops 106 other than the proximal-most loop and the distal-most loop is not smaller than the diameter of the proximal-most loop.

In some embodiments, at least a subset of loops 106 are adapted to apply pressure, in a radial direction, to an interior surface of the gastrointestinal tract, such as to the interior surface of esophagus 10.

In some embodiments, when negative pressure is applied to elongate tube 102, loops 106 form a stack, which provides mechanical strength to the coil during application of the negative pressure.

In some embodiments, a longitudinal end of a shape-forming wire of elongate tube 102 (e.g., shape forming wire 128 of FIGS. 3A and 3B) may be blunt, or may be protected by a blunt structure. For example, the end of the wire may be protected by a plastic or silicone outer end, and by a soft tube or soft wire, as a tail.

In some embodiments, fluid-tight lumen 108 is integrally formed with elongate tube 102.

In some embodiments, fluid-tight lumen 108 has at least a portion that overlaps at least a portion of elongate tube 102.

In some embodiments, elongate tube 102 and fluid-tight lumen 108 are substantially concentric, such that elongate axes thereof are substantially coincidental. However, the coil axis 107 is typically not coincidental with the elongate axis of fluid-tight lumen 108.

In some embodiments, the shape-forming wire (e.g., 128 of FIGS. 3A and 3B) of elongate tube 102 and monofilament 111 of fluid-tight lumen 108 are formed of the same material. In some embodiments, the shape-forming wire and the monofilament are formed of nitinol.

In some embodiments, the shape-forming wire (e.g., 128 of FIGS. 3A and 3B) of elongate tube 102 and monofilament 111 of fluid-tight lumen 108 are a continuous monofilament wire.

In some embodiments, the shape-forming wire (e.g., 128 of FIGS. 3A and 3B) of elongate tube 102 and monofilament 111 of fluid-tight lumen 108 are formed of two different materials.

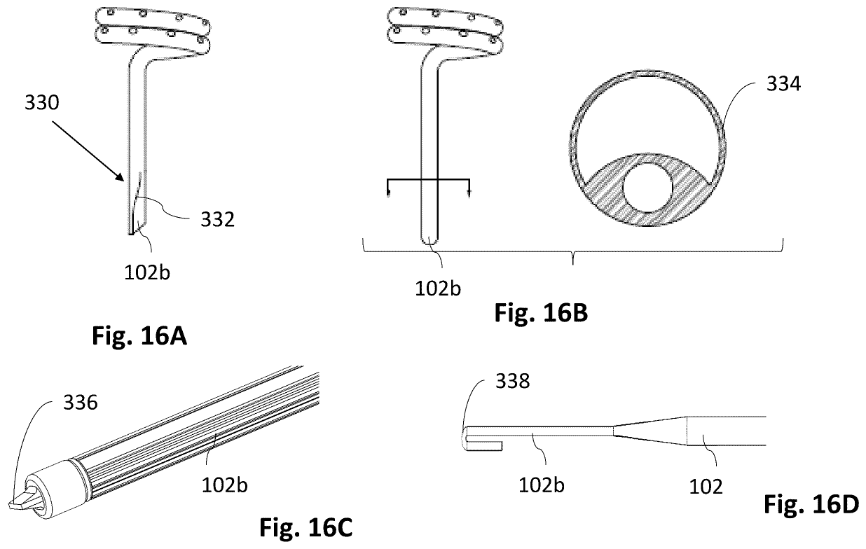

Reference is now made to FIGS. 16A to 16D, which are schematic illustrations of embodiments of valves disposed at a distal end of elongate tube 102 forming part of the medical system of FIGS. 1A and 1B, according to embodiments of the disclosed technology.

As seen, in some embodiments, distal end 102b of elongate tube 102 is open (i.e., not sealed), and a valve is disposed at the distal end 102b. The valve is adapted to be open when no negative pressure is applied to elongate tube 102, for example to facilitate passage of a guidewire through distal end 102b of the tube during deployment thereof, and to be sealed when negative pressure is applied to the elongate tube, in order to ensure that the negative pressure is applied via orifices 104 and not via distal end 102b.

FIG. 16A shows a valve 330, formed of an elastomeric material disposed at end 102b, the elastomeric material including a slit 332.

FIG. 16B shows a valve 332 that is integral in end 102b, and is formed by reducing the thickness of the material of wall 334 of elongate tube 102, so as to ensure that at distal end 102b, wall 334 would collapse when negative pressure is applied to the elongate tube, thereby sealing the distal end of the tube.

FIG. 16C shows a duck-bill valve 336, as known in the art, disposed at the distal end of elongate tube 102.

FIG. 16D shows the distal end 102b of elongate tube 102 being flattened relative to the rest of the tube, and including a 180-degree bend 338. Bend 338 can be straightened when desired, for example when a wire is pushed through the elongate tube and out of the distal end thereof, thus forming the open position of the valve. When the bend 338 is present, the distal tip of elongate tube 102 is sealed, thus forming the closed position of the valve.

It is to be appreciated that any other type of valve may be used at distal end 102b of the elongate tube, including, for example, a cross-slit valve, an elastomeric valve, and the like.

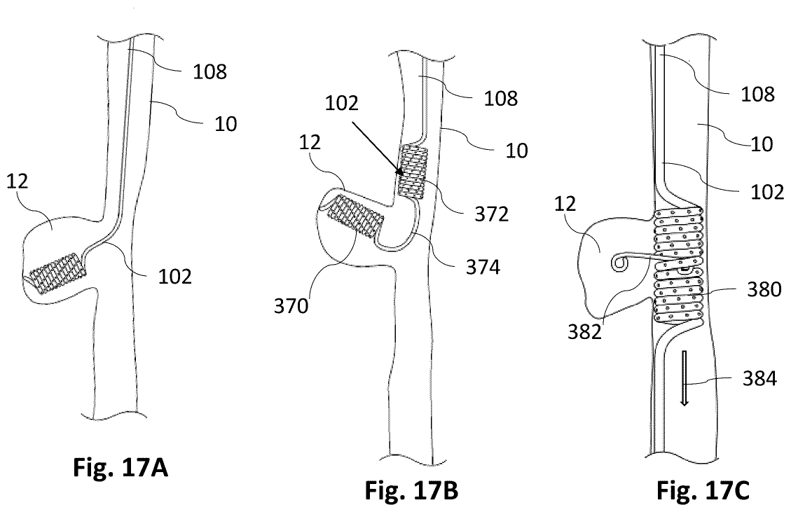

Reference is now made to FIGS. 17A, 17B, and 17C, which are schematic illustrations of embodiments of placement of portions of medical system 100 of FIGS. 1A and 1B, within the gastrointestinal tract, in order to treat a wound 12 in the gastrointestinal tract, according to embodiments of the disclosed technology. In the illustrated embodiments, the wound 12 is an extraluminal wound formed in the wall of esophagus 10. However, such a wound may be formed in any other portion of the gastrointestinal tract, and treated in a similar manner.

In FIG. 17A, a coil formed by elongate tube 102 of medical system 100 is placed within extraluminal wound 12, and is adapted to drain debris and liquid from the wound. Elongate tube 102 is connected to fluid tight lumen 108, which extends intraluminally through esophagus 10, and is adapted to deliver negative pressure to the coil disposed within wound 12.

In FIG. 17B, elongate tube 102 of medical system 100 forms a first, distal, coil 370 which is placed within extraluminal wound 12, and a second, proximal, coil 372 which is disposed intraluminally within esophagus 10, upstream of wound 12. Coils 370 and 372 are in fluid communication with each other. Elongate tube 102 is connected to fluid tight lumen 108, which extends intraluminally through esophagus 10, upstream of coil 372, and is adapted to deliver negative pressure to coils 372 and 370, substantially as described hereinabove.

In some embodiments, and as illustrated, coils 370 and 372 are connected by an un-coiled segment 374 of elongate tube 102.

When negative pressure is applied to elongate tube 102, coil 370 is adapted to drain debris and liquid from the wound, while coil 372 is adapted to drain debris and liquid from the esophagus, so as to assist in maintaining the wound clean and dry, for example when the subject swallows and fluid flows down the esophagus.

In FIG. 17C, a coil 380 formed by elongate tube 102 of medical system 100 is placed intraluminally, within esophagus 10, in an area adjacent wound 12. An additional tube, such as a pigtail 382, extends from coil 380 into wound 12. Negative pressure applied to the vicinity of coil 380 is delivered, via pigtail 382, to the interior of wound 12, to assist in draining of debris and liquid from the wound.

It is to be appreciated that, in some embodiments, a distal end 102b of elongate tube 102 may continue downstream from the location of wound 12, toward the stomach of the subject, for example as indicated by arrow 384 in FIG. 17C. This may be advantageous, for example, when the elongate tube includes a fluid-delivery channel, such as fluid delivery channel 136 of FIG. 9, in which case the fluid delivery channel may be utilized to deliver fluid nourishment into the stomach of the subject during the healing period of the wound.

In some other embodiments, an additional nourishment tube (not explicitly shown) may extend through the axial cavity in the center of coil 380 toward the stomach of the subject, in order to provide nourishment to the subject during the healing period of the wound.

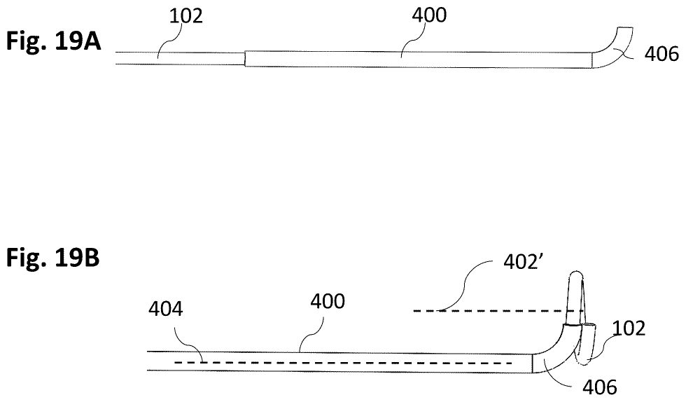

Reference is now made to FIGS. 18A, 18B and 18C, which are schematic illustrations of steps of deploying the medical system 100 into the gastrointestinal tract, for example into esophagus 10 according to an embodiment of the disclosed technology, and to FIGS. 19A and 19B, which are schematic illustrations of a modification of the steps of FIGS. 18A to 18C.

As seen in FIG. 18A, elongate tube 102 is disposed within a lumen of a tubular sheath 400, which functions as a delivery mechanism for delivering elongate tube 102 into the gastrointestinal tract. As such, in the embodiment of FIGS. 18A to 19B, elongate tube 102 is delivered into the gastrointestinal tract of the subject within the lumen of tubular sheath 400. In the embodiment of FIGS. 18A to 19B, elongate tube 102 may be associated with a wire (similar to wire 128 or 138 of FIGS. 3A to 9) which is delivered into the gastrointestinal tract of the subject within the lumen of tubular sheath 400. In this case, the wire is adapted to act as a coil shaped frame for the elongate tube 102 when disassociated from the tubular sheath 400.

Elongate tube 102 is in a delivery state when associated with the tubular sheath 400 and obtains the resting operative state, in which the elongate tube is coiled, when dissociated from the sheath. Tubular sheath 400 is adapted to be removed from the gastrointestinal tract following delivery of elongate tube 102.

Tubular sheath 400 functions as a linearizing element for elongate tube 102, such that, in the delivery state, when the elongate tube is within sheath 400, elongate tube 102 is substantially linear and adapted for delivery, for example via a lumen of a working channel.

In some embodiments, elongate tube 102 is adapted to be removed from the gastrointestinal tract via the lumen of tubular sheath 400. In such embodiments, elongate tube 102 is pulled into the tubular sheath from the proximal end of the tube, and assumes the delivery state during removal thereof from the gastrointestinal tract.

In some embodiments, the elongate tube 102 and the linearizing element, such as tubular sheath 400, are adapted to be separated from one and other by rotation and/or axial motion of elongate tube relative to the linearizing element.

In some embodiments, during deployment of the elongate tube, elongate tube 102 is adapted to form the coil sequentially as segments of the elongate tube are pushed distally out of tubular sheath 400, in the direction of arrow 401, as clearly seen by comparison of FIGS. 18B and 18C. As seen in FIGS. 18B and 18C, elongate tube 102 is pushed distally out of sheath 400, such that the distal end of the elongate tube forms the first, distal loop 106′, shown in FIG. 18B. Subsequently, as additional portions of elongate tube 102 are pushed out the sheath, they form additional loops 106, as shown in FIG. 18C.

In an inverse manner, during removal of elongate tube 102 from the gastrointestinal tract via sheath 400, each of loops 106 is adapted to transform into a substantially linear segment upon entry into the sheath, sequentially, from the proximal loop to the distal loop.

In some embodiments, a distal end of tubular sheath 400 includes a pointed shape. For example, the pointed shape may be desirable in order to pass through debris or through a wound scab on the way to a target destination of the elongate tube.

As seen in FIG. 18C, in some embodiments, a longitudinal axis 402 of the coil is angled (e.g. at a 90-degree angle) with respect to a longitudinal axis 404 of a remainder of elongate tube 102, for example disposed within sheath 400, or coupled (e.g., via the fluid-tight lumen) to the source of negative pressure 110 (FIG. 1A). In some embodiments it is desirable for the longitudinal axes 402 and 404 to be parallel.

In some embodiments, and as shown in FIGS. 19A and 19B, tubular sheath 400 may have a shoulder 406 disposed at a distal end thereof. Shoulder 406 is adapted to redirect elongate tube 102 prior to coiling thereof. As such, and as shown in FIG. 19B, when the coil is formed, longitudinal axis 402′ of the coil is parallel to longitudinal axis 404 of the linear portion of sheath 400, which houses the linear remainder of elongate tube 102.

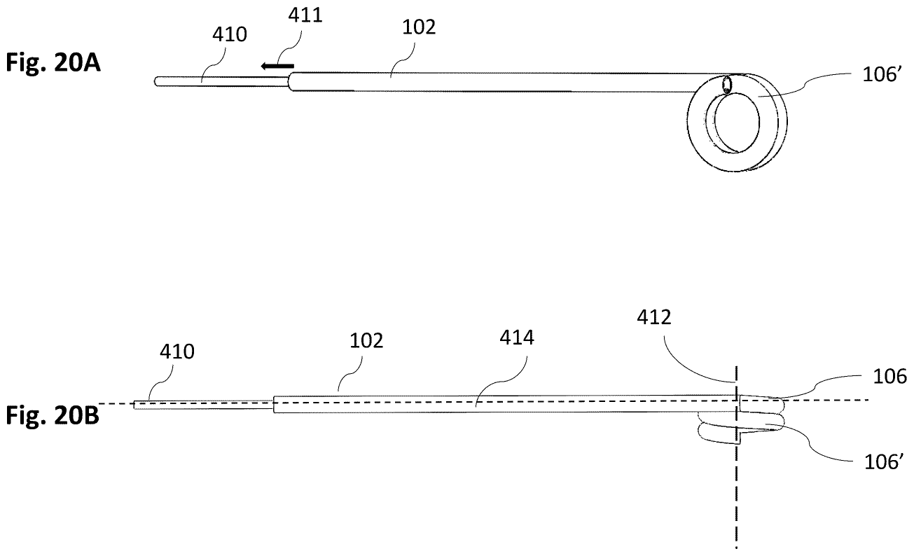

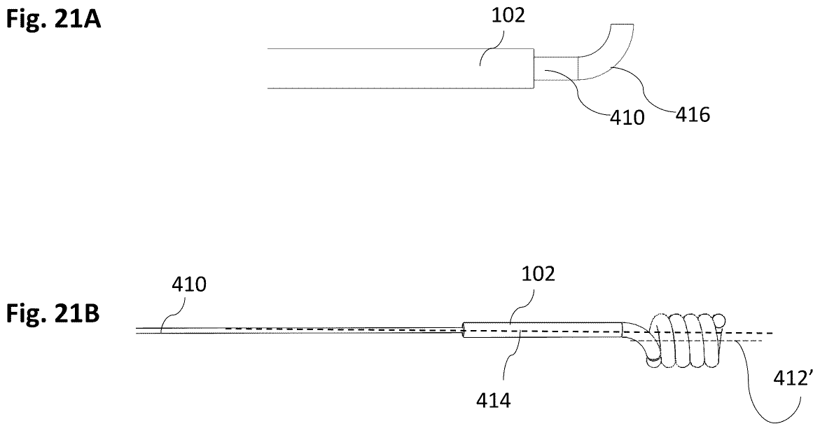

Reference is now made to FIGS. 20A and 20B, which are schematic illustrations of steps of deploying the medical system 100 into the gastrointestinal tract, for example into esophagus 10, according to an embodiment of the disclosed technology, and to FIGS. 21A and 21B, which are schematic illustrations of a modification of the steps of FIGS. 20A to 20B.

As seen in FIG. 20A, a wire 410, which may be a guidewire, is disposed within a channel of elongate tube 102, such as channel 123 (FIG. 3A). Wire 410 is adapted to be delivered into the gastrointestinal tract together with elongate tube. As such, in the embodiment of FIGS. 20A to 21B, elongate tube 102 is delivered into the gastrointestinal tract of the subject with wire 410 extending longitudinally and internally therethrough, and the wire is removed from elongate tube 102 upon delivery thereof into the gastrointestinal tract. In some embodiments, wire 410 may extend through a channel within elongate tube, distinct from the channel used for connection to the source of negative pressure 110 (FIG. 1A). For example, wire 410 may extend through a fluid-delivery channel (e.g. channel 136 of FIG. 9) or through a dedicated channel.

It is to be appreciated that wire 410 is distinct from a shape-forming wire of elongate tube 102 (such as shape forming wire 128 (FIG. 3A)) and from a monofilament of fluid-tight lumen 108 connected to the elongate tube (such as monofilament 111 (FIG. 2)). In some embodiments, when wire 410 is disposed within fluid-tight lumen 108 and/or within elongate tube 102, the wire 410 may be disposed in parallel to the shape-forming wire of the elongate tube and/or the monofilament of the fluid-tight lumen.

In some embodiments, elongate tube 102 is in a delivery state when associated with wire 410 and obtains the resting operative state, in which the elongate tube is coiled, when dissociated from the wire. Wire 410 is adapted to be removed from the gastrointestinal tract following delivery of elongate tube 102, by pulling the wire in the direction of arrow 411, shown in FIG. 20A. In this case, wire 410 functions as a linearizing element for elongate tube 102, such that, in the delivery state, when wire 410 is within the elongate tube, elongate tube 102 is substantially linear. In some embodiments, elongate tube 102 is adapted to form the coil sequentially as segments of wire 410 are extracted proximally out of the elongate tube in the direction of arrow 411, as clearly seen by comparison of FIGS. 20A and 20B. For example, elongate tube 102 may include a shape-forming wire, similar to wire 128 of FIGS. 3A and 3B, which extends through or is embedded within the elongate tube and is adapted to form the coil. As seen in FIGS. 20A and 20B, guidewire 410 is pulled proximally out of elongate tube 102, such that the distal end of the elongate tube forms the first, distal loop 106′, shown in FIG. 20A. Subsequently, as additional portions of elongate tube 102 are dissociated from wire 410 which is pulled out of those portions, they form additional loops 106, as shown in FIG. 20B.

As seen in FIG. 20B, in some embodiments, a longitudinal axis 412 of the coil is angled (e.g. at a 90-degree angle) with respect to a longitudinal axis 414 of a remainder of elongate tube 102, for example having wire 410 disposed therein, or connected to the source of negative pressure 110 (FIG. 1A). In some embodiments it is desirable for the longitudinal axes 412 and 414 to be parallel.

In some embodiments, and as shown in FIGS. 21A and 21B, wire 410 may have a shoulder 416 at a distal end thereof. Shoulder 416 is adapted to redirect elongate tube 102 prior to coiling thereof. As such, and as shown in FIG. 21B, when the coil is formed, longitudinal axis 412′ of the coil is parallel to longitudinal axis 414 of the linear portion elongate tube 102.

It is to be appreciated that, in some embodiments, elongate tube may be delivered into the gastrointestinal tract within a working channel of a delivery device. In some such embodiments, elongate tube 102 may be delivered within the working channel together with a linearizing element, such as sheath 400 (FIGS. 18A to 19B) or guidewire 410 (FIGS. 20A to 21B). In such embodiments, elongate tube 102 (and the associated linearizing element) is sized and configured to pass through the working channel of the delivery device.

In some embodiments, the delivery device may be a catheter or an endoscope.

In some embodiments, the delivery device may include an image capturing element, adapted to provide images of elongate tube 102 during delivery thereof into the gastrointestinal tract.

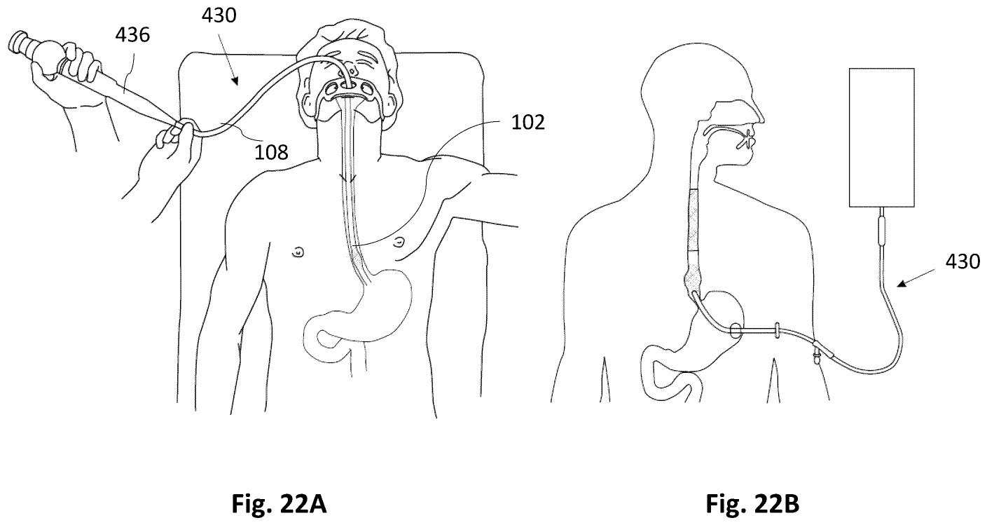

Reference is now made to FIGS. 22A and 22B, which are schematic illustrations of a procedure of deploying medical system 430, similar to medical system 100 including any one of elongate tubes 102, 122, 122a, 122b, 122c, 132, 142, 152, 162, 192, 202, or 212 into the body of a subject, according to embodiments of the disclosed technology.

As seen in FIG. 22A, system 430, and specifically the elongate tube thereof, may be fed into the gastrointestinal tract of the subject, via the subject's mouth. For example, in the illustrated embodiment, an endoscope is used to deliver the elongate tube into the esophagus of the subject, via the subject's mouth. However, it is to be appreciated that the method of FIGS. 22A and 22B may be used for delivery of the system to other parts of the gastrointestinal tract.

In some embodiments, the elongate tube can be disposed within a working channel of a delivery device (not explicitly shown), during delivery thereof into the gastrointestinal tract. As such, in the embodiment of FIG. 22A, the delivery state of elongate tube 102 is a linear state.

In some embodiments, once elongate tube 102 is delivered to the desired location within the gastrointestinal tract (e.g., in the vicinity of a wound), the delivery device is adapted to be retracted to allow the elongate tube to form the coil.

In some embodiments, delivery device may be a catheter or an endoscope.

In some embodiments, delivery device may include an image capturing element (not explicitly shown), adapted to provide images of the elongate tube during delivery thereof into the gastrointestinal tract. For example, the image capturing element may be a video camera adapted to capture images of the interior of the gastrointestinal tract during placement of the elongate tube therein.

In some embodiments, system 100 includes a handle portion 436 mechanically couplable to an end of fluid-tight lumen 108, which is far from elongate tube 102, and which typically remains extracorporeal during deployment of the elongate tube into the gastrointestinal tract. Manipulation of the handle portion, for example by pushing or turning thereof, results in distal motion of elongate tube 102. In some embodiments, the handle portion is adapted to be detached from fluid-tight lumen 108 following delivery of elongate tube 102 into the gastrointestinal tract.

In some embodiments, manipulation of the handle portion causes elongate tube 102 to transition from the delivery state to the coiled, resting operative state. In some embodiments, the transition of elongate tube 102 into the coil is sequential, such that each of loops 106 is adapted to form as the handle portion delivers elongate tube 102 into the gastrointestinal tract, sequentially from the distal loop to the proximal loop, in a similar manner to that shown in FIGS. 18A to 21B.

In FIG. 22B, the elongate tube of system 430 is delivered into the esophagus surgically. Specifically, a hole is punctured in the abdominal wall of the subject, and the elongate tube is delivered to the gastrointestinal tract, and specifically to the esophagus of the subject, via the stomach. Alternatively, a hole is punctured in the submental triangle (not shown) of the subject, and the elongate tube is delivered to the gastrointestinal tract and specifically to the esophagus of the subject via the punctured hole. In some such applications, a distal end of the elongate tube may be sharp, or may include a needle, suitable for puncturing the required hole in the abdominal wall. In some other embodiments, the process may be similar to that of placing a percutaneous endoscopic gastronomy (PEG) device, with the distinction that the elongate tube could be delivered further than the stomach, into the esophagus.

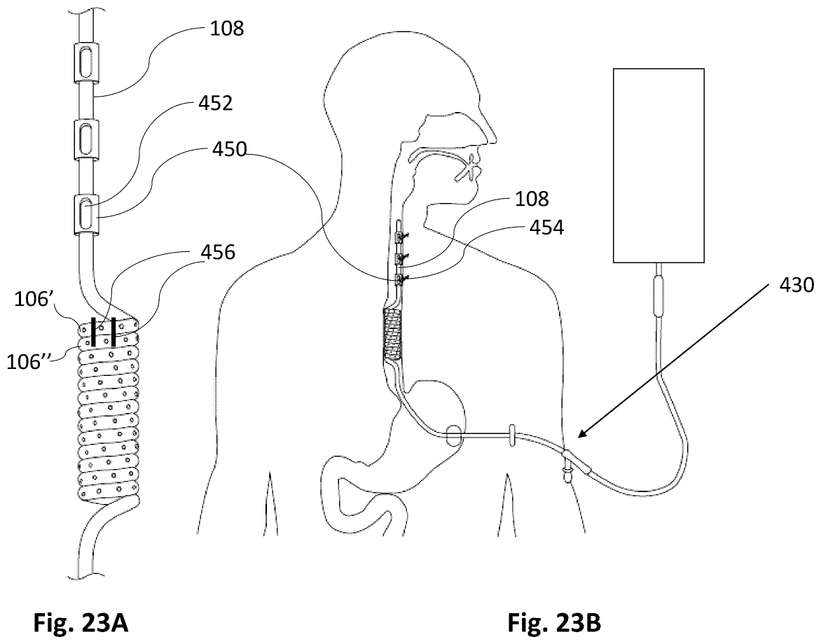

Reference is now additionally made to FIGS. 23A and 23B, which are schematic illustrations of anchoring the medical system of FIGS. 1A and 1B within the gastrointestinal tract according to an embodiment of the disclosed technology. For clarity, the description of FIGS. 23A and 23B is provided with respect to the reference numerals used hereinabove for FIG. 22B, in which the medical system is numbered 430.

As seen in FIG. 23A, fluid tight lumen 108 may have a plurality of anchoring points 450 disposed therealong. Anchoring points 450 typically include a portal or eyelet 452, suitable for threading a tether or suture therethrough. As seen in FIG. 23B, when system 430 is inserted into the esophagus of the user, fluid tight lumen 108 is anchored to the wall of the esophagus, by sutures or tethers 454 extending through each of eyelets 452.

As shown in FIG. 23A, in some embodiments, additional tethers 456 may be used to connect loops 106′ and 106″, closest to fluid tight lumen 108, to each other, to assist in preventing excessive extension of the coil, particularly when external pressure is applied thereto, such as during peristatic motion of the esophagus.

Anchoring of the fluid tight lumen 108 as shown in FIGS. 23A and 23B is particularly useful when the medial system is deployed via the stomach, as shown in FIG. 23B. In such embodiments, fluid tight lumen 108 does not extend through the nasal cavity, since in this case the fluid tight lumen is not held in place by the natural curvature of the space through which it extends, and cannot be held in place externally by clipping the fluid tight lumen to the nose of the user, or the like.

Reference is now made to FIGS. 24A, 24B, 24C, 24D, and 24E, which are schematic illustrations of steps of a procedure for maintaining medical system 430, such as medical system 100 including any one of elongate tubes 102, 122, 122a, 122b, 122c, 132, 142, 152, 162, 192, 202, or 212 in the body of the subject via a nasal wire or tube, following its introduction as shown in FIG. 22A. For brevity, the description relates to elongate tube 102 of medical system 430, relating to the elongate tube of FIGS. 1A and 1B. However, any of the other elongate tubes described hereinabove can replace elongate tube 102. It is to be appreciated that the method of FIGS. 24A and 24E may be used for delivery of the system to other parts of the gastrointestinal tract.

As seen, in FIG. 24A a delivery device 720 is deployed into the esophagus of the subject, via the subject's mouth 740. For example, the delivery device may be the delivery device described hereinabove with respect to FIG. 22A, such as an endoscope. In FIG. 24B, at least part of fluid-tight lumen 108, as well as elongate tube 102, are delivered to the esophagus of the user using delivery device 720 (not shown). A linearizing element is removed from elongate tube 102, such that the elongate tube obtains the coil of its resting operative orientation, and the delivery device is removed from the mouth of the subject, leaving elongate tube 102 and fluid-tight lumen 108, in place. Following placement of medical system 730 within the esophagus of the subject, delivery device 720 is removed from the body, as seen in FIG. 24C.

In FIG. 24D, a wire 750 is inserted into the nose of the subject, and through the sinuses of the subject into, and out of, the subject's mouth. A proximal end of fluid-tight lumen 108 is then associated with end 752 of wire 750 extending out of the subject's mouth. In FIG. 24E, wire 750 is pulled out of the subject's nose. As wire 750 is pulled out of the subject's nose, end 752 of the wire, together with the proximal end of fluid tight lumen 108, are pulled into the subject's nose. When wire 750 is fully removed from the subject's nose, fluid tight lumen 108 continues to extend through the subject's nose, connecting elongate tube 102 to the exterior of the subject's body, for connection to the source of negative pressure 110. Fluid tight lumen 108 can additionally be associated with a nasal retaining element which is configured to maintain the longitudinal position of the coil within the subject.

The disclosed technology may be better understood with respect to the following exemplary embodiments:
1. A medical system for applying negative pressure within a gastrointestinal tract of a subject, the system including:
(a) a linearizing element;
(b) an elongate tube, including:
   (i) at least one channel along at least a longitudinal portion of the elongate tube;
   (ii) at least one portal in fluid communication with the at least one channel; and
   (iii) a shape-forming wire fixed to or embedded within the elongate tube and extending along a longitudinal length thereof,
   the elongate tube having a delivery state when associated with the linearizing element and a first operative state when dissociated from the linearizing element in which the elongate tube forms a coil including a plurality of loops, the coil having an axial length (L) of at least 15 mm and the plurality of loops including at least four loops; and
(c) a fluid-tight lumen in fluid communication with a first end of the elongate tube, the fluid tight lumen being adapted to couple to a source of negative pressure and to deliver negative pressure to the elongate tube via the end of the elongate tube, the fluid-tight lumen comprising:
   (i) a tube formed of a first material; and
   (ii) a longitudinally extending monofilament, formed of a second material, fixed to the tube or embedded therein.
2. A medical system for applying negative pressure within a gastrointestinal tract of a subject, the system including:
(a) a linearizing element;
(b) an elongate tube, including:
   (i) at least one channel along at least a longitudinal portion of the elongate tube;
   (ii) at least one portal in fluid communication with the at least one channel; and
   (iii) a shape-forming wire fixed to or embedded within the elongate tube and extending along a longitudinal length thereof,
   the elongate tube having a delivery state when associated with the linearizing element and a first operative state when dissociated from the linearizing element in which the elongate tube forms a coil including a plurality of loops, the coil having an axial length (L) of at least 15 mm and the plurality of loops including at least four loops,
   wherein the elongate tube has a first flexure modulus in a coil-radial direction of the elongate tube and a second flexure modulus in a coil-axial direction of the elongate tube, the second flexure modulus being greater than the first flexure modulus; and
(c) a fluid-tight lumen in fluid communication with a first end of the elongate tube, the fluid tight lumen being adapted to couple to a source of negative pressure and to deliver negative pressure to the elongate tube via the end of the elongate tube.
3. The medical system of example 2, wherein the fluid-tight lumen includes:
   (i) a tube formed of a first material; and
   (ii) a longitudinally extending monofilament, formed of a second material, fixed to the tube or embedded therein,
   wherein the monofilament has a lower elongation ability than the tube, and
   wherein the monofilament has a tensile modulus greater than 150 Mpa, and
   wherein a flexure modulus of the fluid-tight lumen is smaller than 300 Mpa.
4. A medical system for applying negative pressure within a gastrointestinal tract of a subject, the system including:
(a) a linearizing element;
(b) an elongate tube, including:
   (i) at least one channel along at least a longitudinal portion of the elongate tube;
   (ii) at least one portal in fluid communication with the at least one channel; and
   (iii) a shape-forming wire fixed to or embedded within the elongate tube and extending along a longitudinal length thereof,
   the elongate tube having a delivery state when associated with the linearizing element and a first operative state when dissociated from the linearizing element in which the elongate tube forms a coil including a plurality of loops, the coil having an axial length (L) of at least 15 mm and the plurality of loops including at least four loops; and
(c) a fluid-tight lumen in fluid communication with a first end of the elongate tube, the fluid tight lumen being adapted to couple to a source of negative pressure and to deliver negative pressure to the elongate tube via the end of the elongate tube.
5. A medical system for applying negative pressure within a gastrointestinal tract of a subject, the system including:
(a) a linearizing element;
(b) an elongate tube, including:
   (i) at least one channel along at least a longitudinal portion of the elongate tube; and
   (ii) at least one portal in fluid communication with the at least one channel,
   the elongate tube having a delivery state when associated with the linearizing element and a first operative state when dissociated from the linearizing element in which the elongate tube forms a coil including a plurality of loops, the coil having an axial length (L) of at least 15 mm and the plurality of loops including at least four loops; and
   a fluid-tight lumen in fluid communication with a first end of the elongate tube, the fluid tight lumen being adapted to couple to a source of negative pressure and to deliver negative pressure to the elongate tube via the end of the elongate tube, the fluid-tight lumen comprising:
   (i) a tube formed of a first material; and
   (ii) a longitudinally extending monofilament, formed of a second material, fixed to the tube or embedded therein,
   wherein the monofilament has a lower elongation ability than the tube,
   wherein the monofilament has a tensile modulus greater than 150 Mpa, and
   wherein a flexure modulus of the fluid-tight lumen is smaller than 300 Mpa.
6. A medical system for applying negative pressure within a gastrointestinal tract of a subject, the system including:
   a linearizing element;
   an elongate tube defining at least one channel along at least a longitudinal portion thereof and including at least one portal in fluid communication with the at least one channel, the elongate tube having a delivery state when associated with the linearizing element, and a first operative state when dissociated from the linearizing element in which the elongate tube forms a coil including a plurality of loops, wherein a first portion of the coil is coiled in a first direction and a second portion of the coil is coiled in a second direction, the first portion being connected to the second portion by a shoulder in the coil, and longitudinal axes of the first and second portions of the coil substantially coincide; and
   a fluid-tight lumen in fluid communication with a first end of the elongate tube, the fluid-tight lumen adapted to couple to a source of negative pressure and to deliver negative pressure to the elongate tube via the end of the elongate tube.
7. A medical system for applying negative pressure within a gastrointestinal tract of a subject, the system including:
   a linearizing element;
   an elongate tube defining at least one channel along at least a longitudinal portion thereof and including at least one portal in fluid communication with the at least one channel, the elongate tube having a delivery state when associated with the linearizing element, and a first operative state when dissociated from the linearizing element in which the elongate tube forms a coil including a plurality of loops; and
   a fluid-tight lumen in fluid communication with a first end of the elongate tube, the fluid-tight lumen adapted to couple to a source of negative pressure and to deliver negative pressure to the elongate tube via the end of the elongate tube,
   wherein, each of the plurality of loops of the coil includes a pair of hemispherical portions separated by a substantially linear portion, such that in a top plan view of the loop, the loop has a first dimension and a second dimension, the first and second dimensions being orthogonal to each other, and the first dimension being greater than the second dimension.
8. A medical system for applying negative pressure within a gastrointestinal tract of a subject, the system including:
   a linearizing element;
   an elongate tube defining at least one channel along at least a longitudinal portion thereof and including at least one portal in fluid communication with the at least one channel, the elongate tube having a delivery state when associated with the linearizing element, and a first operative state when dissociated from the linearizing element in which the elongate tube forms a coil including a plurality of loops; and
   a fluid-tight lumen in fluid communication with a first end of the elongate tube, the fluid-tight lumen adapted to couple to a source of negative pressure and to deliver negative pressure to the elongate tube via the end of the elongate tube,
   wherein, each of the plurality of loops of the coil includes a pair of hemispherical portions separated by a substantially linear portion, such that an angle between each loop of the plurality of loops and a longitudinal coil axis extending through the center of the coil is in the range of 45-85 degrees.
9. The medical system of example 8, wherein, for each of the plurality of loops, in a top plan view of the loop, the loop has a first dimension and a second dimension, the first and second dimensions being orthogonal to each other, and the first dimension being greater than the second dimension.
10. A medical system for applying negative pressure within a gastrointestinal tract of a subject, the system including:
   a linearizing element;
   an elongate tube defining at least one channel along at least a longitudinal portion thereof and including at least one portal in fluid communication with the at least one channel, the elongate tube having a delivery state when associated with the linearizing element, and a first operative state when dissociated from the linearizing element in which the elongate tube forms a coil including a plurality of loops, the coil having a length of at least 15 mm and the plurality of loops including at least four loops; and
   a fluid-tight lumen in fluid communication with a first end of the elongate tube, the fluid-tight lumen adapted to couple to a source of negative pressure and to deliver negative pressure to the elongate tube via the end of the elongate tube,
   wherein, when the elongate tube is deployed in the gastrointestinal tract of a landrace female swine having a weight in the range of 60-90 kg, mechanical characteristics of the coil formed by the elongate tube prevents total coil elongation greater than 100%, during at least 48 hours, under natural peristaltic forces within the gastrointestinal tract
11. The medical system of any one of examples 1-10, wherein the fluid-tight lumen is adapted to deliver negative pressure greater than 40 mmHg, 60 mmHg, or 80 mmHg.
12. The medical system of any one of examples 1-11, wherein the fluid-tight lumen is adapted to deliver negative pressure smaller than 350 mmHg, 300 mmHg, or 250 mmHg.
13. The medical system of any one of examples 1-12, wherein the fluid-tight lumen is adapted to deliver negative pressure in the range of 40 mmHg to 350 mmHg, 60 mmHg to 300 mmHg, or 80 mmHg to 250 mmHg.
14. The medical system of any one of examples 1-13, wherein the at least one channel of the elongate tube is adapted to remain open when the negative pressure is applied to the elongate tube.
15. The medical system of any one of examples 1-14, wherein the at least one portal includes a plurality of orifices.
16. The medical system of any one of examples 1-14, wherein the at least one portal includes at least one slot extending longitudinally along the elongate tube.
17. The medical system of any one of examples 5-16, wherein the elongate tube includes a shape-forming wire fixed to or embedded within the elongate tube and extending along a longitudinal length thereof.
18. The medical system of any one of examples 1-5 or 17, the elongate tube has a first flexure modulus in a coil-radial direction of the elongate tube and a second flexure modulus in a coil-axial direction of the elongate tube, the second flexure modulus being greater than the first flexure modulus.
19. The medical system of example 18, wherein the second flexure modulus in the coil-axial direction is at least twice as large as the first flexure modulus in the coil-radial direction.
20. The medical system of example 18 or example 19, wherein the first flexure modulus in the coil radial direction is in the range of 20 Mpa to 3000 Mpa.
21. The medical system of example 18 or example 19, wherein the first flexure modulus in the coil radial direction is in the range of 20 Mpa to 1000 Mpa.
22. The medical system of any one of examples 18 to 21, wherein the second flexure modulus in the coil-axial direction is greater than 500 Mpa.
23. The medical system of any one of examples 18 to 22, wherein, for a cross section of the elongate tube, a radial moment of inertia of the elongate tube is smaller than an axial moment of inertia of the elongate tube.
24. The medical system of any one of examples 1-5 or 17-23, wherein a relative rotational orientation between the shape-forming wire and the elongate tube remains fixed along the length of the entire elongate tube.
25. The medical system of any one of examples 1-5 to or 17-24, wherein the shape-forming wire is disposed in the same position along the entire longitudinal length of the elongate tube, such that in a longitudinal cross-section of the coil:
   for a coil having a fixed pitch, the vertical distance between each pair of adjacent sections of the shape-forming wire is substantially fixed; and
   for a coil having a non-fixed pitch, the vertical distance between each pair of adjacent sections of the shape-forming wire is substantially equal to the sum of pitch of the coil between the pair of adjacent sections and an exterior diameter of the elongate tube.
26. The medical system of any one of examples 1-5 or 17-25, wherein the shape-forming wire is disposed in the same position along the entire longitudinal length of the elongate tube, such that in a longitudinal cross-section of the coil, in each cross section of the elongate tube, the relative positions of the at least one channel and of the shape-forming wire are substantially the same.
27. The medical system of any one of examples 1-5 or 17-26, wherein the shape-forming wire is disposed in the same position along the entire longitudinal length of the elongate tube, such that in a longitudinal cross-section of the coil, all the shape-forming wire sections are disposed along two straight parallel lines.
28. The medical system of any one of examples 1-5 or 17-26, wherein, within the coil, a distance between the shape-forming wire and the longitudinal axis of the coil is fixed.
29. The medical system of any one of examples 1-5 or 17-28, wherein a cross-section of the shape-forming wire perpendicular to the longitudinal axis of the elongate tube has a first aspect and a second aspect, the first aspect being greater than the second aspect.
30. The medical system of any one of examples 1-5 or 17-29, wherein a greatest dimension of a cross-section of the shape-forming wire, perpendicular to the longitudinal axis of the elongate tube, is smaller than 2.0 mm.
31. The medical system of example 30, wherein the greatest dimension is smaller than 1.5 mm.
32. The medical system of example 30, wherein the greatest dimension is smaller than 1.2 mm.
33. The medical system of example 30, wherein the greatest dimension is smaller than 1.0 mm.
34 The medical system of example 30, wherein the greatest dimension is smaller than 0.8 mm.
35. The medical system of any one of examples 1-5 or 17-34, wherein a smallest dimension of a cross-section of the shape-forming wire, perpendicular to the longitudinal axis of the elongate tube, is greater than 0.1 mm.
35. The medical system of example 34, wherein the smallest dimension is greater than 0.2 mm.
36. The medical system of example 34, wherein the smallest dimension is greater than 0.3 mm.
37. The medical system of example 34, wherein the smallest dimension is greater than 0.4 mm.
38. The medical system of example 34, wherein the smallest dimension is greater than 0.5 mm.
39. The medical system of example 34, wherein the smallest dimension is greater than 0.7 mm.
40. The medical system of any one of examples 1-5 or 17-39, wherein the shape-forming wire has an elastic range greater than 0.5%.
41. The medical system of any one of examples 1-5 or 17-40, wherein the shape-forming wire is formed of nitinol.
42. The medical system of any one of examples 1-5 and 17-41, wherein the shape-forming wire is adapted to extend through the length of the elongate tube at a first time, the medical system further including a second shape-forming wire adapted to extend through the length of the elongate tube at a second time, wherein the first and second shape forming wires having different mechanical characteristics, such that the coil has different mechanical characteristics at the first time than at the second time.
43. The medical system of any one of examples 1-5 and 17-42, wherein the elongate tube additionally includes a wire-channel adapted to accommodate the shape-forming wire.
44. The medical system of example 42, wherein the elongate tube additionally includes a first wire-channel adapted to accommodate the shape-forming wire and a second wire-channel adapted to accommodate the second shape-forming wire.
45. The medical system of example 42, wherein the elongate tube additionally includes a single wire-channel adapted to accommodate the both the shape-forming wire and the second shape-forming wire.
46. The medical system of example 42, wherein the shape-forming wire and the second shape-forming wire are adapted to be accommodated within the at least one channel of the elongate tube.
47. The medical system of any one of examples 1-5 or 17-46, wherein the shape-forming wire is an elastic wire, a super-elastic wire, or a shape memory wire.
48. The medical system of any one of examples 1-5 or 17-47, wherein the shape-forming wire is tubular.
49. The medical system of any one of examples 1-5 or 17-47, wherein the shape-forming wire is a flat wire.
50. The medical system of any one of examples 1-5 or 17-47, wherein the shape-forming wire has a circular, oval, or D-shaped cross section, in a direction perpendicular to a longitudinal axis of the shape-forming wire.
51. The medical system of any one of examples 1-5 or 17-47, wherein the shape-forming wire has a polygonal cross section, in a direction perpendicular to a longitudinal axis of the shape-forming wire.
52. The medical system of any one of examples 1-5 or 17-51, wherein at least one end of the shape-forming wire is protected by a blunt cover.
53. The medical system of any one of examples 1-52, wherein the cross section of the elongate tube, in a direction perpendicular to the longitudinal axis of the elongate tube is round.
54. The medical system of any one of examples 1-53, wherein the linearizing element is a guidewire, the guidewire being adapted to be disposed within the elongate tube and the fluid-tight lumen during delivery of the elongate tube into the gastrointestinal tract of the subject, and to be removed from the elongate tube upon delivery of the elongate tube into the gastrointestinal tract of the subject.
55. The medical system of example 54, wherein the guidewire is distinct from the shape-forming wire.
56. The medical system of any one of examples 1-55, wherein the linearizing element comprises a tubular sheath defining a lumen, and the elongate tube is adapted to be delivered into the gastrointestinal tract, in the delivery state, within the lumen of the tubular sheath.
57. The medical system of any one of examples 1-56, wherein the linearizing element and the elongate tube, in the delivery state, are adapted to be delivered into the gastrointestinal tract of the subject via a working channel of a delivery tool.
58. The medical system of any one of examples 4 or 6-57, wherein the fluid-tight lumen includes:
   (i) a tube formed of a first material; and
   (ii) a longitudinally extending monofilament, formed of a second material, fixed to the tube or embedded therein.
59. The medical system of example 58, wherein the monofilament has a lower elongation ability than the tube.

60. The medical system of example 58 or example 59, wherein the monofilament has a tensile modulus greater than 150 Mpa.
61. The medical system of any one of examples 58-60, wherein a flexure modulus of the fluid-tight lumen is smaller than 300 Mpa.
62. The medical system of any one of examples 1, 3, 5, or 58-61, wherein the flexure modulus of the fluid-tight lumen is smaller than 200 Mpa.
63. The medical system of any one of examples 1, 3, 5, or 58-61, wherein the flexure modulus of the fluid tight lumen is smaller than 100 Mpa.
64. The medical system of any one of examples 1, 3, 5, or 58-63, wherein the fluid-tight lumen has a minimal bending radius of 15 cm or more, without forming kinks in the fluid-tight lumen.
65. The medical system of any one of examples 1, 3, 5, or 58-64, wherein the monofilament is embedded in the tube of the fluid-tight lumen.
66. The medical system of any one of examples 1, 3, 5, or 58-64, wherein the monofilament is fixed to the tube of the fluid-tight lumen, along a longitudinal length thereof.
67. The medical system of any one of examples 1-66, wherein, when a force of 10N is applied axially to the fluid-tight lumen, an elongation percentage of the fluid-tight lumen is not greater than 5%.
68. The medical system of any one of examples 1-66, wherein, when a force of 10N is applied axially to the fluid-tight lumen, an elongation percentage of the fluid-tight lumen is not greater than 2%.
69. The medical system of any one of examples 1-66, wherein, when a force of 10N is applied axially to the fluid-tight lumen, an elongation percentage of the fluid-tight lumen is not greater than 1%.
70. The medical system of any one of examples 1-69, wherein the fluid-tight lumen has a tensile modulus greater than 200 Mpa.
71. The medical system of any one of examples 1-69, wherein the fluid-tight lumen has a tensile modulus greater than 300 Mpa.
72. The medical system of any one of examples 1-69, wherein the fluid-tight lumen has a tensile modulus greater than 400 Mpa.
73. The medical system of any one of examples 1-69, wherein the fluid-tight lumen has a tensile modulus greater than 500 Mpa.
74. The medical system of any one of examples 1-73, wherein a tensile modulus of the fluid-tight lumen is greater than the flexure modulus of the fluid-tight lumen.
75. The medical system of any one of examples 1-73, wherein the tensile modulus of the fluid-tight lumen is at least twice as large as the flexure modulus of the fluid-tight lumen.
76. The medical system of any one of claim 1, 3, 5, or 58-75, wherein a tensile modulus of the fluid-tight lumen is greater than a tensile modulus of the tube, and wherein a flexure modulus of the fluid-tight lumen is substantially equal to a flexure modulus of the tube.
77. The medical system of any one of claim 1, 3, 5, or 58-76, wherein a tensile modulus of the fluid-tight lumen substantially equal to the tensile modulus of the monofilament.
78. The medical system of any one of examples 1, 3, 5, or 58-77, wherein the linearizing element is a guidewire, the guidewire being adapted to be disposed within the elongate tube and the fluid-tight lumen during delivery of the elongate tube into the gastrointestinal tract of the subject, and to be removed from the elongate tube upon delivery of the elongate tube into the gastrointestinal tract of the subject.
79. The medical system of example 78, wherein the guidewire is distinct from the monofilament.
80. The medical system of any one of examples 1, 3, 5, or 58-79, wherein each dimension of a cross-section of the monofilament is in the range of 0.1 mm to 1.5 mm.
81. The medical system of any one of examples 1, 3, 5, or 58-79, wherein each dimension of a cross-section of the monofilament is in the range of 0.1 mm to 1.0 mm.
82. The medical system of any one of examples 1, 3, 5, or 58-79, wherein each dimension of a cross-section of the monofilament is in the range of 0.1 mm to 0.8 mm.
83. The medical system of any one of examples 1, 3, 5, or 58-79, wherein each dimension of a cross-section of the monofilament is in the range of 0.1 mm to 0.5 mm.
84. The medical system of any one of examples 1, 3, 5, or 58-83, wherein the shape-forming wire and the monofilament are formed of the same material.
85. The medical system of any one of examples 1, 3, 5, or 58-84, wherein the shape-forming wire and the monofilament form a continuous monofilament wire.
86. The medical system of any one of examples 1, 3, 5, or 58-83, wherein the shape-forming wire and the monofilament are formed of different materials.
87. The medical system of any one of examples 1-86, wherein the shape-forming wire and/or the monofilament are non-absorbable in the human GI tract.
88. The medical system of any one of examples 1-5 or 7-87, wherein a first portion of the coil is coiled in a first direction and a second portion of the coil is coiled in a second direction, the first portion being connected to the second portion by a bend in the coil, and longitudinal axes of the first and second portions of the coil substantially coincide.
89. The medical system of any one of examples 1-6 or 9-88, wherein, when the elongate tube is in the coil form an end section of the elongate tube, distal relative to the fluid tight lumen and having a length greater than an external diameter of the coil, remains linear.
90. The medical system of any one of examples 1-6 or 9-89, wherein, when the elongate tube is in the coil form a proximal end of the coil forms a shoulder in the range of 1-60 degrees relative to a longitudinal axis of the coil.
91. The medical system of any one of examples 1-8 or 10-90, wherein, each of the plurality of loops of the coil includes a pair of hemispherical portions separated by a substantially linear portion, such that in a top plan view of the loop, the loop has a first dimension and a second dimension, the first and second dimensions being orthogonal to each other, and the first dimension being greater than the second dimension.
92. The medical system of any one of examples 1-9 or 11-91, wherein, each of the plurality of loops of the coil includes a pair of hemispherical portions separated by a substantially linear portion, such that an angle between each loop of the plurality of loops and a longitudinal coil axis extending through the center of the coil is in the range of 45-85 degrees.
93. The medical system of any one of examples 6-91, wherein each of the first portion of the coil and the second portion of the coil includes at least three of the plurality of loops.
94. The medical system of any one of examples 13-93, wherein the end section of the elongate tube remains linear or the proximal end of the coil forms the shoulder relative to the longitudinal axis of the coil.
95. The medical system of any one of examples 7-94, wherein the end section is adapted to ensure that a longitudinal axis of the coil is within an angular threshold of a longitudinal axis of a portion of the gastrointestinal tract in which the coil is deployed.
96. The medical system of any one of examples 7-95, wherein the shoulder is adapted to ensure that a first loop of the coil, closest to the fluid tight lumen, has a parallel orientation to other loops of the coil.
97. The medical system of any one of examples 7-96, wherein the shoulder is adapted to ensure that the at least one channel remains unobstructed following coiling of the elongate tube.
98. The medical system of any one of examples 10-97, wherein the angle between each loop of the plurality of loops and the longitudinal coil axis is in the range of 50-75 degrees.
99. The medical system of any one of examples 10-97, wherein the angle between each loop of the plurality of loops and the longitudinal coil axis is in the range of 55-65 degrees.
100. The medical system of any one of examples 1-99, wherein a diameter of a cross sectional area of the coil, perpendicular to a longitudinal axis of the coil, is in the range of 10 mm to 30 mm, and a pitch between loops of the coil is smaller than 15 mm.
101. The medical system of any one of examples 1-100, wherein the elongate tube includes at least one exterior channel extending longitudinally along an exterior surface of the elongate tube between the at least one portal and an end of the elongate tube.
102. The medical system of any one of examples 1-101, wherein the at least one channel extends longitudinally through the interior of the entire elongate tube.
103. The medical system of any one of examples 1-102, wherein the elongate tube is formed of a porous material.
104. The medical system of any one of examples 1-103, wherein the elongate tube has antimicrobial or anti-inflammatory properties.
105. The medical system of example 104, wherein the elongate tube includes an antimicrobial or anti-inflammatory material.
106. The medical system of example 104, wherein the elongate tube is pretreated or coated with at least one antimicrobial or anti-inflammatory agent.
107. The medical system of any one of examples 104-106, wherein the gastrointestinal tract of the subject has an endoluminal wound, and the elongate tube is adapted to deliver to the vicinity of the endoluminal wound an antimicrobial or anti-inflammatory medicament for treatment of the endoluminal wound.
108. The medical system of any one of examples 1-106, wherein the elongate tube has a textured exterior surface adapted to frictionally engage an interior surface of the gastrointestinal tract.
109. The medical system of any one of examples 1-108, wherein at least a portion of the elongate tube is detachable from the fluid-tight lumen.
110. The medical system of example 109, wherein the at least a portion of the elongate tube is formed from a biocompatible material and is adapted to be naturally excreted from the body of the subject following detachment from the fluid-tight lumen.
111. The medical system of any one of examples 1-108, wherein the at least a portion of the elongate tube is formed from a biodegradable material, and is adapted to be degraded or decomposed, within the body of the subject.
112. The medical system of any one of examples 1-111, wherein the at least one channel includes a first channel adapted for drainage of a fluid from the gastrointestinal tract, via the at least one portal, when negative pressure is applied to the elongate tube.
113. The medical system of example 112, wherein the first channel is in fluid communication with a drainage receptacle adapted to receive the fluid drained from the gastrointestinal tract via the first channel.
114. The medical system of example 112, wherein the first channel is in fluid communication with a second location in the gastrointestinal tract, downstream from the location at which the coil is located, and is adapted to release fluid drained from the gastrointestinal tract at the second location.
115. The medical system of any one of examples 1-114, wherein the at least one channel includes a second channel adapted to be coupled to a source of a fluid and to deliver the fluid into a portion of the gastrointestinal tract via the elongate tube and via at least some of the at least one portal.
116. The medical system of example 115, further including the source of the fluid.
117. The medical system of example 115 or example 116, wherein the fluid includes a medicament fluid.
118. The medical system of example 115 or example 116, wherein the fluid includes a contrast fluid.
119. The medical system of example 115 or example 116, wherein the fluid includes ionized gas.
120. The medical system of example 115 or example 116, wherein the fluid includes carbon dioxide.
121. The medical system of any one of examples 1-120, wherein the fluid-tight lumen and the elongate tube are substantially concentric.
122. The medical system of any one of examples 1-121, wherein the elongate tube includes a shape memory material, an elastic material, or a super-elastic material adapted to form the coil in the first operative state.
123. The medical system of example 122, wherein the super elastic material or shape memory material is a shape memory alloy, a spring alloy, a polymer or nitinol.
124. The medical system of example 122 or example 123, wherein the elongate tube consists of the shape memory material.
125. The medical system of any one of examples 15 or 17-124, wherein the plurality of orifices are disposed about a single circumference of the elongate tube.
126. The medical system of any one of examples 15 or 17-124, wherein the plurality of orifices are disposed longitudinally along a longitudinal length of the elongate tube.
127. The medical system of any one of examples 15 or 17-126, wherein the plurality of orifices are equidistantly distributed along or about the elongate tube.
128. The medical system of any one of examples 15 or 17-127, wherein the orifices are heterogeneously distributed along or about the elongate tube.
129. The medical system of any one of examples 15 or 17-127, wherein the each of the plurality of orifices has substantially the same diameter.
130. The medical system of any one of examples 15 or 17-127, wherein orifices in a first subset of the plurality of orifices have a first diameter, and orifices in a second subset of the plurality of orifices have a second diameter, the second diameter being different from the first diameter.
131. The medical system of any one of examples 15 or 17-130, wherein, in the first operative state, at least some of the plurality of orifices are oriented inwardly, toward a center of the coil.
132. The medical system of any one of examples 15 or 17-131, wherein, in the first operative state, at least some of the plurality of orifices are oriented outwardly, away from a center of the coil.
133. The medical system of any one of examples 15 or 17-131, wherein, in the first operative state, the coil is substantially devoid of orifices oriented outwardly, away from a center of the coil.
134. The medical system of any one of examples 16-133, wherein the at least one slot includes at least one slot extending along the entire longitudinal length of the elongate tube.
135. The medical system of any one of examples 16-133, wherein the at least one slot includes a plurality of slots, equidistantly distributed about a circumference of the elongate tube.
136. The medical system of any one of examples 16-133, wherein the at least one slot includes at least one slot extending along a portion of the longitudinal length of the elongate tube.
137. The medical system of any one of examples 1-136, wherein the plurality of loops includes at least two loops having substantially the same diameter.
138. The medical system of example 137, wherein the at least two loops include a proximal-most loop and a distal-most loop of the plurality of loops.
139. The medical system of example 138, wherein the diameter of one of the plurality of loops other than the proximal-most loop and the distal-most loop is not greater than the diameter of the proximal-most loop.
140. The medical system of example 138, wherein the diameter of one of the plurality of loops other than the proximal-most loop and the distal-most loop is not smaller than the diameter of the proximal-most loop.
141. The medical system of example 137, wherein the at least two loops having substantially the same diameter includes a first loop and a second loop, and wherein a distance between the first loop and the second loop, along a longitudinal axis of the coil, is greater than 10 mm.
142. The medical system of any one of examples 1-141, wherein in the first operative state of the elongate tube, the plurality of loops includes at least 4 loops.
143. The medical system of any one of examples 1-141, wherein in the first operative state of the elongate tube, the plurality of loops includes at least 5 loops.
144. The medical system of any one of examples 1-141, wherein in the first operative state of the elongate tube, the plurality of loops includes at least 8 loops.
145. The medical system of any one of examples 1-141, wherein in the first operative state of the elongate tube, the plurality of loops includes at least 10 loops.
146. The medical system of any one of examples 1-145, wherein in the first operative state of the elongate tube, a number of loops in the plurality of loops is within a range of 4 to 15.
147. The medical system of any one of examples 1-145, wherein in the first operative state of the elongate tube, a number of loops in the plurality of loops is within a range of 5 to 15.
148. The medical system of any one of examples 1-145, wherein in the first operative state of the elongate tube, a number of loops in the plurality of loops is within a range of 5 to 12.
149. The medical system of any one of examples 1-145, wherein in the first operative state of the elongate tube, a number of loops in the plurality of loops is within a range of 8 to 12.
150. The medical system of any one of examples 1-149, wherein, in the first operative state of the elongate tube, a diameter of the coil is in a range of 0.5 cm to 5 cm.
151. The medical system of any one of examples 1-150, wherein, in the first operative state of the elongate tube, a diameter of the coil is in a range of 1 cm to 5 cm.
152. The medical system of any one of examples 1-150, wherein, in the first operative state of the elongate tube, a diameter of the coil is in a range of 0.5 cm to 4 cm.
153. The medical system of any one of examples 1-150, wherein, in the first operative state of the elongate tube, a diameter of coil is in a range of 1 cm to 3 cm.
154. The medical system of any one of examples 1-150, wherein, in the first operative state of the elongate tube, a diameter of the coil is in a range of 2.0 cm to 4.0 cm.
155. The medical system of any one of examples 1-150, wherein, in the first operative state of the elongate tube, a diameter of the coil is in a range of 2.5 cm to 3.5 cm.
156. The medical system of any one of examples 1-155, wherein, in the first operative state of the elongate tube, a diameter of the coil is not greater than 1.5 cm.
157. The medical system of any one of examples 1-155, wherein, in the first operative state of the elongate tube, a diameter of the coil is not greater than 1.5 cm.
158. The medical system of any one of examples 1-157, wherein in the first operative state of the elongate tube, an axial length of the coil is at least 15 mm.
159. The medical system of any one of examples 1-157, wherein in the first operative state of the elongate tube, an axial length of the coil is at least 20 mm.
160. The medical system of any one of examples 1-159, wherein in the first operative state of the elongate tube, an axial length of the coil is at most 200 mm.
161. The medical system of any one of examples 1-159, wherein in the first operative state of the elongate tube, an axial length of the coil is at most 150 mm.
162. The medical system of any one of examples 1-159, wherein in the first operative state of the elongate tube, an axial length of the coil is at most 100 mm.
163. The medical system of any one of examples 1-159, wherein in the first operative state of the elongate tube, an axial length of the coil is at most 80 mm.
164. The medical system of any one of examples 1-159, wherein in the first operative state of the elongate tube, an axial length of the coil is at most 70 mm.
165. The medical system of any one of examples 1-159, wherein in the first operative state of the elongate tube, an axial length of the coil is at most 60 mm.
166. The medical system of any one of examples 1-159, wherein in the first operative state of the elongate tube, an axial length of the coil is at most 50 mm.
167. The medical system of any one of examples 1-166, wherein, when a negative pressure of 80-250 mmHg is applied to the elongate tube, a second axial length of the coil is in the range of 10 mm to 50 mm.
168. The medical system of any one of examples 1-166, wherein, when a negative pressure of 80-250 mmHg is applied to the elongate tube, a second axial length of the coil is in the range of 10 mm to 40 mm.
169. The medical system of any one of examples 1-166, wherein, when a negative pressure of 80-250 mmHg is applied to the elongate tube, a second axial length of the coil is in the range of 20 mm to 50 mm.
170. The medical system of any one of examples 1-166, wherein, when a negative pressure of 80-250 mmHg is applied to the elongate tube, a second axial length of the coil is in the range of 20 mm to 40 mm.
171. The medical system of any one of examples 1-170, wherein at least one characteristic of the coil is configurable by making a change to a condition in an environment surrounding the coil.
172. The medical system of example 171, wherein the at least one characteristic includes a chemical characteristic or a mechanical characteristic of the coil.
173. The medical system of example 171 or example 172, wherein the condition includes a temperature.
174. The medical system of any one of examples 1-173, wherein the linearizing element is adapted to be removed from the gastrointestinal tract of the subject following delivery of the elongate tube.
175. The medical system of any one of examples 1-174, wherein, in the delivery state, the elongate tube is substantially linear.
176. The medical system of any one of examples 1-175, wherein the linearizing element is a tubular sheath defining a lumen, and wherein the elongate tube is adapted to be delivered into the gastrointestinal tract within the lumen.
177. The medical system of example 176, wherein the elongate tube is adapted to be removed from the gastrointestinal tract via the lumen, and to assume the delivery state during the removal.
178. The medical system of example 176 or example 177, and wherein elongate tube is adapted to form the coil sequentially as segments of the elongate tube are pushed distally out of the delivery sheath, from the distal loop to the proximal loop.
179. The medical system of example 176-178, and wherein during removal of the elongate tube from the gastrointestinal tract, each of the loops of the coil is adapted to transform into a substantially linear segment, sequentially, from the proximal loop to the distal loop.
180. The medical system of any one of examples 176-179, wherein a distal end of the tubular sheath includes a removable pointed shape.
181. The medical system of any one of examples 1-175, wherein the linearizing element is a guidewire, and wherein the elongate tube is adapted to be delivered into the gastrointestinal tract with the guidewire extended internally therethrough.
182. The medical system of example 181, wherein the elongate tube includes a first channel in fluid communication with the fluid-tight lumen and a second channel adapted for passage of the guidewire therethrough.
183. The medical system of any one of examples 181-182, wherein the elongate tube is adapted to form the coil sequentially, as the guidewire is retracted proximally out of the elongate tube, from the distal loop to the proximal loop.
184. The medical system of any one of examples 1-183, wherein the linearizing element terminates in a shoulder at an end thereof, the shoulder adapted to control a direction of the longitudinal axis of the coil.
185. The medical system of any one of examples 1-184, further including a handle portion mechanically couplable to a second end of the fluid tight lumen, far from the elongate tube, such that manipulation of the handle portion results in distal motion of the elongate tube.
186. The medical system of example 185, wherein the manipulation includes pushing or turning of the handle portion.
187. The medical system of example 185 or example 186, wherein the handle portion is adapted to be detached from the fluid tight lumen following delivery of the elongate tube into the gastrointestinal tract.
188. The medical system of any one of examples 185-187, wherein the manipulation of the handle portion causes the elongate tube to transition from the delivery state to the first operative state.
189. The medical system of example 188, wherein the transition is sequential, such that each of the plurality of loops is adapted to form as the elongate tube is delivered into the gastrointestinal tract, sequentially from the distal loop to the proximal loop.
190. The medical system of any one of examples 1-189, wherein the fluid-tight lumen, and the elongate tube in the delivery state, are sized and configured to pass through a working channel of a delivery device, wherein the working channel has a diameter or less than 5 mm.
191. The medical system of example 190, wherein an external delivery sheath, adapted to be disposed above the elongate tube during delivery thereof, is sized and configured to pass through the working channel of the delivery device.
192. The medical system of example 191, wherein the delivery device includes a catheter or an endoscope.
193. The medical system of any one of examples 191-192, wherein the delivery device further includes an image capturing element, adapted to provide images of the elongate tube during delivery thereof into the gastrointestinal tract.
194. The medical system of any one of examples 1-193, further including the source of negative pressure functionally associated with the fluid-tight lumen, the source of negative pressure being adapted to apply to the elongate tube a negative pressure in the range of 40-350 mmHg.
195. The medical system of example 194, wherein the source of negative pressure includes a controller adapted to regulate the negative pressure provided by the source of negative pressure, within a pressure range, to remove fluid from the vicinity of at least a portion of the internal surface of the gastrointestinal tract.
196. The medical system of example 195, wherein the elongate tube is adapted to deliver negative pressure to a portion of the gastrointestinal tract including an internal wound, and wherein the controller is adapted to regulate the negative pressure for removal of fluid from the vicinity of an internal surface of the body conduit including the internal wound.
197. The medical system of example 195 or example 196, further including at least one sensor adapted to sense at least one characteristic of the fluid removed from the vicinity of the at least a portion of the gastrointestinal tract, and wherein the controller is adapted to adjust one or more operating parameters of the source of negative pressure in response to input received from the at least one sensor, the information relating to the at least one characteristic of the fluid.
198. The medical system of any one of examples 1-197, further including a fluid source functionally associated with the fluid-tight lumen, and adapted to provide fluid to the elongate tube, via the fluid tight lumen.
199. The medical system of any one of examples 1-198, further including an additional tube, adapted to extend through an internal cavity of the coil.
200. The medical system of any one of examples 1-199, further including a valve, disposed at a distal end of the elongate tube, the valve adapted to be in a closed operative state when the negative pressure is applied to the elongate tube.
201. The medical system of any one of examples 1-200, further including a reinforcing wire extending through a wall of the elongate tube.
202. The medical system of example 201, wherein the reinforcing wire forms a helix within the wall of the elongate tube.
203.

The medical system of example 202, wherein a direction of rotation of the helix is opposite to a direction of rotation of the coil formed by the elongate tube.
204. The medical system of any one of examples 1-203, wherein the coil includes a first coil portion and a second coil portion, in fluid communication with each other, wherein longitudinal axes of the first and second coil portions are angled with respect to each other.
205. The medical system of example 204, wherein, when the coil is deployed within the gastrointestinal tract, the first portion of the coil is adapted to be disposed intraluminally to the gastrointestinal tract, and the second portion of the coil is adapted to be disposed extraluminally to the gastrointestinal tract.
206. A method of applying negative pressure to a portion of the gastrointestinal tract of a subject, the method including:
(a) placing within the gastrointestinal tract of the subject an elongate tube defining at least one channel along at least a longitudinal portion thereof and having at least one portal in fluid communication with the at least one channel, the elongate tube forming a coil including a plurality of loops, an end of the elongate tube being in fluid communication with a first end of a fluid-tight lumen, wherein a vicinity of the elongate tube, within the gastrointestinal tract, is in fluid communication with the at least one channel via the at least one portal;
(b) coupling a second end of the fluid-tight lumen to a source of negative pressure; and
(c) while the elongate tube is in the form of the coil within the gastrointestinal tract, applying negative pressure to the gastrointestinal tract via the elongate tube and the fluid-tight lumen, wherein, within the gastrointestinal tract, a length of the coil is at least 15 mm and the plurality of loops includes at least four loops.
207. The method of example 206, wherein the placing includes placing the elongate tube such that the coil or a portion thereof, is disposed within an extraluminal wound extending from the gastrointestinal tract, and at least a portion of the fluid tight lumen extends intraluminally within the gastrointestinal tract.
208. The method of example 206, wherein the placing includes placing the elongate tube such that the coil or a portion thereof, is disposed adjacent an intraluminal wound and at least a portion of the fluid tight lumen extends intraluminally within the gastrointestinal tract.
209. The method of example 206, wherein the placing includes placing the elongate tube such that the coil or a portion thereof, is disposed within an extraluminal wound extending from the gastrointestinal tract, and at least a portion of the fluid tight lumen extends intraluminally within the gastrointestinal tract.
210. The method of example 206, wherein the elongate tube forms a first coil portion and a second coil portion, the first and second coil portions being connected by a shoulder portion of the elongate tube and being in fluid communication, and wherein the placing includes placing the elongate tube such that the first coil is disposed within an extraluminal wound extending from the gastrointestinal tract, and the second coil is disposed intraluminally within the gastrointestinal tract.
211. The method of example 206, wherein the placing includes placing the elongate tube such that the coil is disposed intraluminally within the gastrointestinal tract in the vicinity of an extraluminal wound extending from the gastrointestinal tract, the method further including placing a pigtail in fluid communication with the coil and with the extraluminal wound, the pigtail adapted to deliver negative pressure from the elongate tube to the extraluminal wound for draining thereof.
212. The method of any one of examples 206-211, wherein the placing includes delivering the elongate tube within a tubular sheath into the gastrointestinal tract, and the forming includes advancing the elongate element distally out of the tubular sheath.
213. The method of any one of examples 206-212, wherein the placing includes advancing a linearizing wire into the elongate tube and delivering the elongate tube together with the linearizing wire into the gastrointestinal tract, and the forming includes retracting the linearizing wire proximally relative to the elongate tube.
214. The method of any one of examples 206-213, wherein the forming includes forming the plurality of loops sequentially, from the distal loop to the proximal loop.
215. The method of any one of examples 206-214, further including delivering a fluid into the gastrointestinal tract via the fluid-tight lumen, the elongate tube, and the at least one portal.
216. A method of delivering a medical system into a portion of the gastrointestinal tract of a subject, the method including:
(a) delivering (e.g., orally, rectally, percutaneously and preferably orally) a delivery-state elongate tube and a fluid-tight lumen, associated with a linearizing element, into the gastrointestinal tract of the subject, such that the elongate tube is disposed at a target location within the gastrointestinal tract of the subject and a second end of the fluid-tight lumen, distal to the elongate tube, remains outside the mouth of the subject; and
(b) removing the linearizing element from the elongate tube and the fluid-tight lumen, thereby to allow the elongate tube to form the coil within the target location in the gastrointestinal tract of the subject.
217. A method of treating a subject, the method including:
(a) delivering a medical system into a portion of the gastrointestinal tract of a subject according to the method of example 216;
(b) connecting the fluid-tight lumen to a negative pressure source;
(c) applying negative pressure in the range of 25-350 mmHg to the fluid tight lumen;
(d) maintaining the elongate tube within the body of the subject for a predetermined treatment duration; and
(e) following completion of the predetermined treatment duration, removing the elongate tube from the body of the subject.
218. The method of example 217, further including, prior to (c), transitioning the fluid-tight lumen from the oral cavity of the subject to a nasal cavity of the subject, and wherein the removing includes removing the elongate tube via the nose of the subject.
219. The method of example 216, further including, following (b), transitioning the fluid-tight lumen from the oral cavity of the subject to a nasal cavity of the subject.
220. The method of example 219, further comprising retaining elongate tube within the gastrointestinal tract of the subject, and the fluid-tight lumen within the nasal cavity of the subject, for a duration of at least 48 hours.
221. The method of any one of examples 216 or 219-220, further comprising, following (c):
(d) coupling a second end of the fluid-tight lumen to a source of negative pressure; and
(e) while the elongate tube is in the form of the coil within the gastrointestinal tract, applying negative pressure to the gastrointestinal tract via the elongate tube and the fluid-tight lumen.
222. The method of any one of examples 216-221, wherein the delivering includes delivering the elongate tube such that the coil or a portion thereof, will be disposed within an extraluminal wound extending from the gastrointestinal tract, and at least a portion of the fluid-tight lumen extends intraluminally within the gastrointestinal tract.
223. The method of any one of examples 216-221, wherein the delivering includes delivering the elongate tube such that the coil or a portion thereof, will be disposed adjacent an intraluminal wound and at least a portion of the fluid tight lumen extends intraluminally within the gastrointestinal tract.
224. The method of any one of examples 216-221, wherein the delivering includes delivering the elongate tube such that the coil or a portion thereof will be disposed within an extraluminal wound extending from the gastrointestinal tract, and at least a portion of the fluid tight lumen extends intraluminally within the gastrointestinal tract.
225. The method of any one of examples 216-221, wherein the elongate tube is adapted to form a first coil portion and a second coil portion, the first and second coil portions being connected by a shoulder portion of the elongate tube and being in fluid communication, and wherein the delivering includes delivering the elongate tube such that the first coil will be disposed within an extraluminal wound extending from the gastrointestinal tract, and the second coil will be disposed intraluminally within the gastrointestinal tract.
226. The method of any one of examples 216-221, wherein the delivering includes delivering the elongate tube such that the coil will be disposed intraluminally within the gastrointestinal tract in the vicinity of an extraluminal wound extending from the gastrointestinal tract, the method further including placing a pigtail in fluid communication with the coil and with the extraluminal wound, the pigtail adapted to deliver negative pressure from the elongate tube to the extraluminal wound for draining thereof.

It should be understood that the terms “approximately” and “substantially” are defined to include any value within 5% of the approximate value.

It should be understood that the use of “and/or” is defined inclusively such that the term “a and/or b” should be read to include the sets: “a and b,” “a or b,” “a,” “b.”

The various systems, devices, apparatuses, etc. in this disclosure can be sterilized (e.g., with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, for some applications, sterilizing one or more of any of the various systems, devices, apparatuses, etc. in this disclosure.

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver gastrointestinal tract, simulator (e.g., with the body parts, tissue, etc. being simulated), etc.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

1. A medical system for applying negative pressure within a gastrointestinal tract of a subject, the system comprising:
(a) a linearizing element;
(b) an elongate tube, including:
(i) at least one channel along at least a longitudinal portion of the elongate tube;
(ii) at least one portal in fluid communication with the at least one channel; and
(iii) a shape-forming wire fixed to or embedded within the elongate tube and extending along a longitudinal length thereof,
the elongate tube having a delivery state when associated with the linearizing element and a first operative state when dissociated from the linearizing element in which the elongate tube forms a coil including a plurality of loops, the coil having an axial length (L) of at least 15 mm and the plurality of loops including at least four loops,
wherein a rotational orientation of said shape-forming wire relative to said elongate tube is fixed throughout an entire length of said shape-forming wire; and
(c) a fluid-tight lumen in fluid communication with a first end of the elongate tube, the fluid tight lumen being adapted to couple to a source of negative pressure and to deliver negative pressure to the elongate tube via the end of the elongate tube.

2. The medical system of claim 1, wherein a cross-section of said shape-forming wire, in a direction perpendicular to a longitudinal axis of the shape-forming wire, has a first aspect and a second aspect, the first aspect being larger than the second aspect.

3. The medical system of claim 2, wherein:
said elongate tube includes, as said at least one channel, a vacuum-delivery channel and a wire-channel, said vacuum-delivery channel and said wire-channel extending alongside one another;
said shape-forming wire is accommodated with said wire channel such that said first aspect of said shape-forming wire is substantially aligned with a height of said vacuum delivery channel and with a diameter of said elongate tube, throughout said entire length of said shape-forming wire.

4. The medical system of claim 1, wherein, when said elongate tube is linearized, a position of said shape-forming wire relative to an external circumference of said elongate tube remains fixed throughout the entire length of said shape-forming wire.

5. The medical system of claim 1, wherein a cross-section of said elongate tube, in a direction perpendicular to a longitudinal axis of said elongate tube, is asymmetrical along at least one axis of the cross-section, and wherein, when associated with said linearizing element, said elongate tube is devoid of rotation about said longitudinal axis of said elongate tube.

6. The medical system of claim 1, wherein the elongate tube has a first flexure modulus in a coil-radial direction of the elongate tube and a second flexure modulus in a coil-axial direction of the elongate tube, the second flexure modulus being greater than the first flexure modulus.

7. The medical system of claim 6, wherein the second flexure modulus in the coil-axial direction is at least twice as large as the first flexure modulus in the coil-radial direction.

8. The medical system of claim 6, wherein the first flexure modulus in the coil-radial direction is in the range of 20 Mpa to 3000 Mpa or 20 Mpa to 1000 Mpa.

9. The medical system of claim 6, wherein the second flexure modulus in the coil-axial direction is greater than 500 Mpa.

10. The medical system of claim 6, wherein, for a cross section of the elongate tube, a radial moment of inertia of the elongate tube is smaller than an axial moment of inertia of the elongate tube.

11. The medical system of claim 1, wherein said elongate tube is adapted to form said coil within an esophagus of the subject, upon dissociation from said linearizing element.

12. The medical system of claim 1, wherein the fluid-tight lumen includes:
(i) a tube formed of a first material; and
(ii) a longitudinally extending monofilament, formed of a second material, fixed to the tube or embedded therein,
wherein the monofilament has a lower elongation ability than the tube, and
wherein the monofilament has a tensile modulus greater than 150 Mpa, and
wherein a flexure modulus of the fluid-tight lumen is smaller than 300 Mpa.

13. The medical system of claim 1, wherein, in the first operative state, at least some of the plurality of orifices are oriented inwardly, toward a center of the coil.

14. The medical system of claim 1, wherein, in the first operative state, the coil is substantially devoid of orifices oriented outwardly, away from a center of the coil.

15. A method of treating a portion of the gastrointestinal tract of a subject using the medical system of claim 1 by applying negative pressure thereto, the method comprising:
(a) delivering the elongate tube associated with the linearizing element and in the delivery state, as well as a portion of the fluid-tight lumen, into the gastrointestinal tract of the subject, such that the elongate tube is disposed at a target location within the gastrointestinal tract of the subject and a second end of the fluid-tight lumen, distal to the elongate tube, remains outside the body of the subject;
(b) removing the linearizing element from the elongate tube and the fluid-tight lumen, thereby to allow the elongate tube to form the coil within the target location in the gastrointestinal tract of the subject;
(c) connecting the fluid-tight lumen to a negative pressure source;
(d) applying negative pressure to the fluid tight lumen;
(e) maintaining the elongate tube within the body of the subject for a predetermined treatment duration; and
(f) following completion of the predetermined treatment duration, removing the elongate tube from the body of the subject.

16. The method of claim 15, further comprising, prior to (d), transitioning the fluid-tight lumen from an oral cavity of the subject to a nasal cavity of the subject, and
wherein the removing comprises removing the elongate tube via the nose of the subject.

17. The method of claim 15, wherein the predetermined treatment duration is at least 48 hours.
